(19) United States
(12) Patent Application Publication
(10) Pub. No.: US 2025/0177300 A1
(43) Pub. Date: Jun. 5, 2025

(54) ACELLULAR COMPOSITIONS AND METHODS

(71) Applicants: WR BIOTECH II LLC, Morristown, NJ (US); Rutgers, The State University of New York, New Brunswick, NJ (US)

(72) Inventors: Bobak Shadpoor, Newark, NJ (US); Pranela Rameshwar, Maplewood, NJ (US)

(21) Appl. No.: 19/052,229

(22) Filed: Feb. 12, 2025

Related U.S. Application Data
(63) Continuation of application No. 18/485,458, filed on Oct. 12, 2023, now abandoned.
Application No. 18/485,458 is a continuation of application No. PCT/US2023/072820, filed on Aug. 24, 2023.
(60) Provisional application No. 63/400,643, filed on Aug. 24, 2022.

(51) Int. Cl. A61K 9/127; A61K 35/15; A61K 35/28; A61P 39/00

(52) U.S. Cl. CPC A61K 9/127; A61K 35/15; A61K 35/28; A61P 39/00

(57) ABSTRACT

Allogenic, acellular compositions comprising extracellular vesicles derived from pre-primed hematopoietic stem cells and more particularly, pre-primed umbilical cord cells are disclosed. The extracellular vesicles comprise microvesicles, exosomes or a combination thereof. Methods of using such compositions to revitalize cells, tissue and organs, as well as methods of making such compositions are also provided.

### FIELD OF THE INVENTION

Disclosed herein is an acellular, allogenic compositions (e.g., pharmaceutical compositions) comprising extracellular vesicles (ECVs) (e.g., microsomes, exosomes) derived from the secretome of pre-primed hematopoietic cells, including hematopoietic stem cells (HSCs) (e.g., umbilical cord blood cells). Also disclosed are methods of using and manufacturing such compositions. In certain embodiments, the composition disclosed herein is useful for revitalizing aging cells and tissue microenvironment, as well as treating certain age-related disease or disorders.

### BACKGROUND

The process of aging is the result of a combination of genetic and environmental factors. Both contribute to the progressive, time-dependent molecular disorder that underlies the loss of structure and function characteristic of aging.

Aging has historically been viewed as a natural and universal process, as opposed to a disease. Yet, aging can be experienced negatively by the host (e.g., changes in appearance, energy) and is also an independent risk factor for multiple diseases and disorders, including cancer, cardiovascular disease and neurodegenerative disorders.

Despite the vast expenditures targeted to both aging as a natural but modifiable process, and to diseases and disorders tied to aging, there remains a need for new solutions. The challenge is only increasing, as the proportion of the world's population over 60 years will nearly double from 12% to 22% between 2015 and 2050.

### SUMMARY

Disclosed herein are compositions suitable for use in modulating the aging process (e.g., altering age-related biology) well as treating or preventing age-related diseases and disorders. Also disclosed are methods of manufacturing such compositions. Advantageously, the compositions are allogeneic without untoward effects, i.e., permit off-the-shelf use.

In a first aspect, an allogeneic, acellular composition is provided comprising at a plurality of extracellular vesicles (ECVs), wherein the ECVs derived from the secretome of a pre-primed biological sample comprising a mixture of hematopoietic cells such as hematopoietic stem cells (HSC).

In one embodiment, the ECVs are selected from microsomes, exosomes or a combination thereof.

In a particular embodiment, the ECVs consist essentially of microsomes, exosomes or a combination thereof.

In one embodiment, the biological sample is umbilical cord blood (UCB), optionally depleted of mature cells (e.g., T-cells).

In one embodiment, the hematopoietic cells are pre-primed by contacting the cells for a time period with a secretome or isolate thereof derived from aged mononuclear cells (MNCs).

In a particular embodiment, the source of the aged MNC is selected from adult peripheral blood (PB), adult plasma, adult mobilized peripheral blood or adult bone marrow.

In a particular embodiment, the time period is between about 36 and 48 hours.

In one embodiment, the composition further comprises one or more additional components selected from the group consisting of peptides, proteins, cytokines, lipids, nucleic acids, hormones or a combination thereof.

In a particular embodiment, the composition is stable for about 7 days or more when stored at 4° C., −20° C. or −80° C.

In a second aspect, the composition comprises a secretome or fraction thereof derived from a biological sample comprising a population of hematopoietic cells, wherein the biological sample is pre-primed by exposure for a time period to an aged secretome or isolate thereof derived from an aged MNC.

In one embodiment, the composition comprises a total secretome.

In another embodiment, the composition consists essentially of a total secretome.

In one embodiment, the composition comprises a total secretome depleted of one or more undesirable components.

In another embodiment, the composition consists essentially of a EVC-enriched fraction of the total secretome.

In a particular embodiment, the ECV-enriched fraction is a MV-enriched fraction.

In another particular embodiment, the ECV-enriched fraction is an exosome-enriched fraction.

In a third aspect, a pharmaceutical composition is provided comprising the composition disclosed herein and a pharmaceutically acceptable carrier, diluent or excipient.

In a fourth aspect, a dosage form is provided comprising the composition or pharmaceutical composition disclosed herein.

In one embodiment, the dosage form is a liquid dosage form. The liquid dosage form may be provided as a liquid or reconstituted prior to use (e.g., from a lyophilized composition).

In a fifth aspect, a kit is provided comprising the composition or pharmaceutical composition disclosed herein.

In a sixth aspect, a method of using the composition or pharmaceutical composition disclosed herein comprising (i) contacting at least one aged cell with the composition.

In one embodiment, the contacting occurs in vitro. In certain embodiments, method comprising contacting aged bone marrow with the composition in vitro.

In another embodiment, the contacting occurs in vivo.

In a seventh aspect, a method of revitalizing a cell, tissue or organ is provided comprising (i) contacting a cell, tissue or organ with an effective amount of the composition or pharmaceutical composition disclosed herein, thereby revitalizing the cell, tissue or organ.

In one embodiment, the contacting is in vitro.

In another embodiment, the contacting is in vivo.

In certain embodiments, the method further comprising (ii) determining the revitalization.

In one embodiment, the cells are lymphocytes and more particularly, T-and B-lymphocytes, and other immune cells.

In one embodiment, the revitalizing comprises a decrease in myeloid cells.

In a particular embodiment, the determining in (ii) comprises identifying a change in one or more biomarkers associated with aging.

In one embodiment, the determining in (ii) comprises measuring a modified proliferation response to antigen and mitogen stimulation; and natural killer activity.

In one embodiment, the determining in (ii) comprising measuring modified cytokine production.

In one embodiment, the determining in (ii) comprises measuring a decrease in inflammatory cytokines and reduced production of other senescence associated factors such as reactive oxygen species.

In an eighth aspect, a method of modulating aging is provided comprising (i) administering an effective amount of the composition or pharmaceutical composition disclosed herein to a subject in need thereof, thereby modulating aging.

In one embodiment, the method of administering is subcutaneous injection.

In one embodiment, the method further comprises (ii) determining the modulation. In a particular embodiment, the determining comprises identifying a change in one or more biomarkers associated with aging.

In one embodiment, the biomarker is selected from a genetic biomarker or a phenotypic biomarker.

In one embodiment, the method further comprising administering at least one additional agent (e.g., an anti-aging agent) to the subject in need thereof.

In a ninth aspect, a method of treating an age-related disease or disorder is provided, comprising (i) administering a therapeutically effective amount of the composition to a subject in need thereof, thereby treating the disease or disorder.

In one embodiment, the age-related disease or disorder is selected from cancer, a cardiovascular disorder, a neurological disorder, an inflammatory disorder or a combination thereof.

In one embodiment, the method further comprises (ii) determining an increase or decrease in at least one biomarker associated with the age-related disease or disorder.

In one embodiment, the biomarker is selected from a genetic biomarker or a phenotypic biomarker.

In one embodiment, the method further comprising administering at least one additional therapeutical agent (e.g., an anti-cancer agent) to the subject in need thereof.

In a tenth aspect, a method is providing for improving immune function comprising (i) administering a therapeutically effective amount of the composition to a subject in need thereof, thereby improving immune function.

In one embodiment, the method further comprises (ii) determining the improvement in immune function.

In a particular embodiment, the determining in (ii) comprises measuring B cell function, T cell function or a combination thereof.

In an eleventh aspect, a method is provided for making the composition disclosed herein comprising (i) providing an aged secretome of a mononuclear cell (MNC) derived from an aged biological sample; (ii) providing a hematopoietic system; and (iii) bringing the aged secretome into contact with the hematopoietic cells for a time period to permit the hematopoietic cells to shed a plurality of extracellular vesicles (e.g., microsomes, exosomes) into the extracellular space (e.g., cell culture media), and (iv) collecting the extracellular vesicles, thereby providing the composition disclosed herein.

In one embodiment, the aged biological sample is aged peripheral blood, aged plasma, aged mobilized peripheral blood or aged bone marrow.

In one embodiment, the hematopoietic cells are isolated or derived from umbilical cord blood (UBC) or placental blood. In certain embodiments, the hematopoietic cells (e.g.,HSCs) are substantially pure.

In one embodiment, the placental cells are isolated or derived from the placenta. Cells that may be derived from the placenta include embryonic-like stem cells, multipotent cells, and committed progenitor cells. In certain embodiments, the placental cells (e.g., placental stem cells) are substantially pure.

In a particular embodiment, the hematopoietic cells are cultured and sheds the ECVs into the cell culture media. The cell culture media is then concentrated, e.g., >200-fold. In certain embodiments, the concentrated media is enriched for ECVs between about 1 and about 1000 nm in size.

In a particular embodiment, the placental cells are cultured and shed the ECVs into the cell culture media. The cell culture media is then concentrated, e.g., >200-fold. In certain embodiments, the concentrated media is enriched for ECVs between about 1 and about 1000 nm in size.

In a particular embodiment, the ECVs are enriched by differential centrifugation.

In a particular embodiment, the MVs are enriched by differential centrifugation

In a particular embodiment, the exosomes are enriched by differential centrifugation.

In one embodiment, the time period is between about 36 and 48 hours.

### BRIEF DESCRIPTION OF THE FIGURES

FIG. 1 depicts the mechanism by which young mobilized peripheral blood (MPB) revitalized the aged hematopoietic system according to the present method. Specifically, the aged samples used in the novel method are aged bone marrow mononuclear cells, aged plasma or aged peripheral blood. The secretome from the aged cells communicate with young umbilical cord blood (UCB) devoid of T-cells through a process referred to as pre-priming. The secretome of the pre-primed UCB cells comprising microvesicles (MVs) represents one embodiment of the final product (optionally, further purified). The efficacy of the final product according to this embodiment is tested by its effects on the aged hematopoietic system (priming), e.g., in vivo.

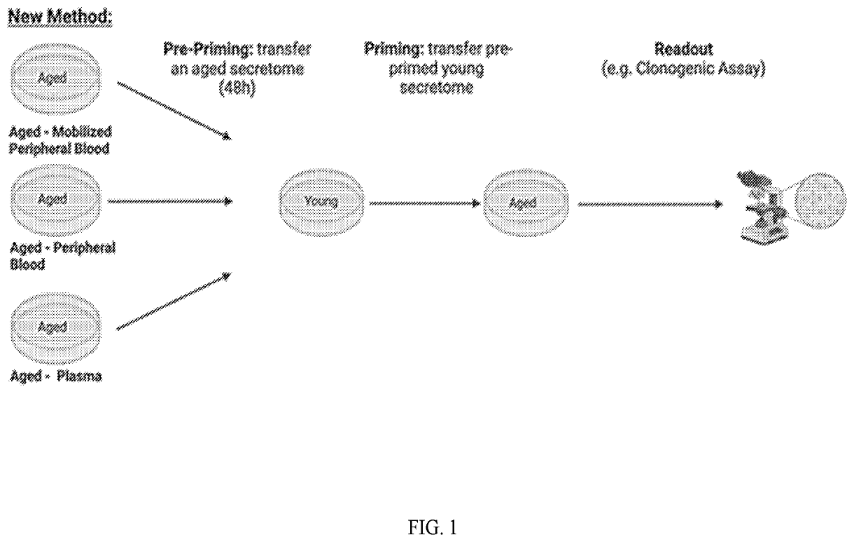

FIG. 2 depicts pre-priming for >48 hours used aged mononuclear cells and UCB. Umbilical Cord Blood (UCB) cells pre-primed with aged secretome for 48 h, 72 h, or 96 h. Pre-Primed secretome applied to aged cells and allowed to undergo revitalization during priming periods of 3, 5, or 7 days. Pre-prime—48 h secretome demonstrated the greatest degree of revitalization. These results prompted the analysis of pre-priming less than 48 hours. Also, it was considered whether hematopoietic activity with different pre-priming time-points could enhance hematopoietic activity.

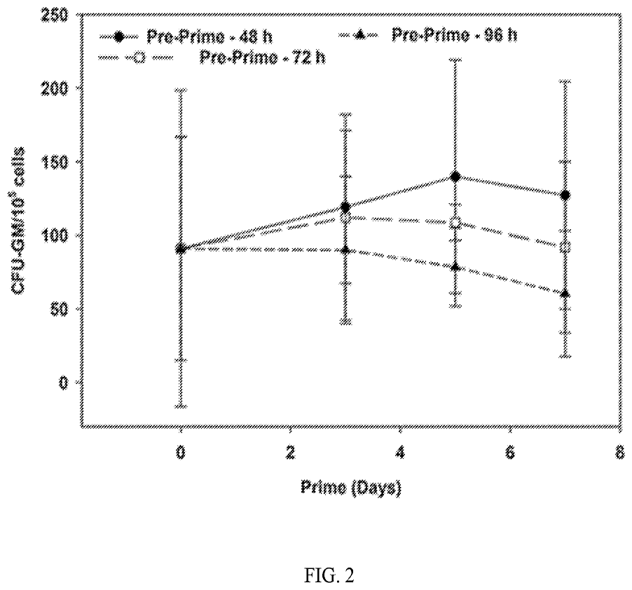

FIG. 3 shows graphs of the pre-priming at 24 h, 48 h, 72 h, and 96 h. Assay was performed as described in FIG. 2.

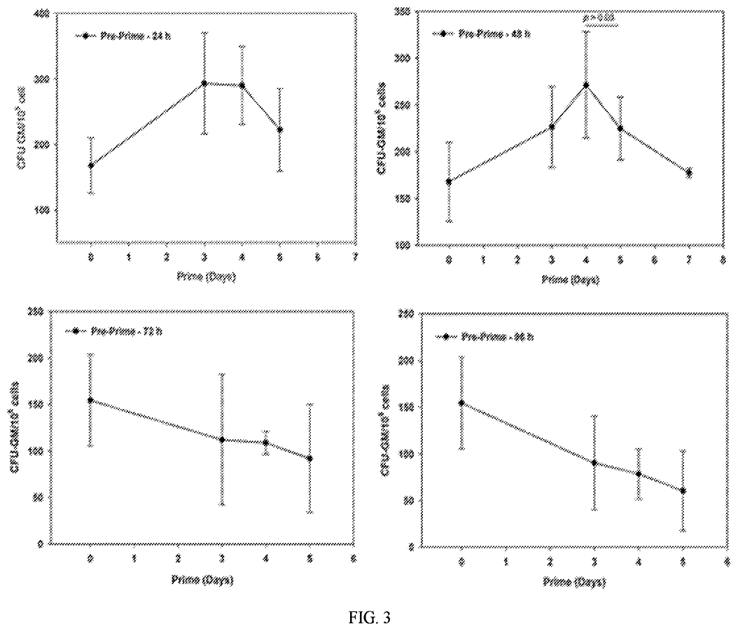

FIG. 4 shows a graph of the clonogenic assay results. Mice (n=2), aged 10 or 20 months, were treated with 10¹⁰ particles of exemplary exosomes. The mice were injected intraperitoneally at Day 0 and Day 3. Mice were euthanized at Day 10. The cells in mice femurs were collected and assayed for colony forming unit (CFU-GM).

FIG. 5 shows a graph of the sex differences in the results of treated mice. Mice (n=1), aged 10 and 20 months, were treated with 10¹⁰ particles of exemplary exosomes. *p<0.05 vs. male mice. **p>0.05 vs. male mice.

FIG. 6 shows images of relative clonogenic size in treated and untreated mice. The images show larger clones of CFU-GM in the treated bone marrow cells from both 10 and 20 month treated mice, as compared untreated mice (represents colonies from untreated 10 and 20 month mice).

FIG. 7 shows a graph of the effect of aged plasma on hematopoietic restoration. Controls were exemplary exosomes using the insoluble fraction alone (i.e., exosomes), or combining the exosomes (insoluble factors released from aged cells), and soluble fraction (factors other than exosomes) from Aged MPBs. Baseline colonies, shown in open bars, include aged MPBs alone (No treatment). The figure shows the mean+SD CFU-GM colonies/105 aged cells.

FIG. 8 shows a graph of the nanoparticles of the exemplary exosomes in each sample after storage with trehalose at various temperatures. Exosomes were resuspended in phosphate buffered saline containing 25 mM Trehalose prior to storage at 4° C., −20° C., and −80° C. Particles were enumerated over the course of three weeks. All particles were confirmed to be 30-150 nm in diameter consistent with exosome size.

FIG. 9 shows a graph of the nanoparticles of the exemplary exosomes in each sample after resuspension in phosphate buffered saline with or without 1% human serum albumin (HSA) prior to storage at 4° C., −20° C., and −80° C. Particles were enumerated over the course of two weeks.

FIG. 10 shows a graph of the nanoparticles of the exemplary exosomes in each sample after resuspension in phosphate buffered saline and stored at 4° C. Particles were enumerated over 4 weeks.

### DETAILED DESCRIPTION

### Definitions

The term “acellular” as used herein refers to compositions with significantly reduced intact cell content. For example, acellular as applicable to plasma may indicate low or no cellular content as compared to commonly available isolated peripheral blood plasma. Acellular compositions or materials may be generated by any means known in the art. In certain embodiments, the acellular compositions disclosed herein are not produced by filtration. In certain embodiments, the acellular compositions disclosed herein may include some intact cells or remnants of cells, however, the therapeutic components or agents within the compositions may be predominantly acellular components.

The term “administering” as used herein includes prescribing for administration as well as administering a composition. It includes physically administering by the subject being treated or by another. Administration can be carried out by any suitable route, including for example oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrastemal, intrathecal, intraperitoneal, intrahepatic, intralesional, and intracranial injections or infusion techniques), and the like.

The term “adult” as used herein means post-embryonic. In one aspect, the stem cells of the invention may be isolated an adult, e.g., a mammal, such as a human. Adult stem cells according to the invention may be isolated from any non-embryonic tissue, and will include neonates, juveniles, adolescents and adult subjects or patients.

The term “adult stem cell” refers to post-natal stem cells derived from tissues, organs or blood of an adult.

The terms “aging” or “senesce” as used herein refers to the gradual loss of function and deterioration at the cellular, tissue, and/or organ level. This gradual loss of homeostasis renders an organism more vulnerable to disease and disability. Aging can be broadly categorized as normal (i.e., universal changes”) or usual (i.e., age-related diseases or disorders not found in all older organisms, such as cancer). Normal aging is characterized, for example, by (i) changes in the biochemical composition of tissue; (ii) progressive decrease in physiological capacity; (iii) diminished ability to respond adaptively to environmental stimuli; and (iv) increased susceptibility and vulnerability to disease (e.g., cancer, cardiovascular disease, stroke, chronic lung disease and infections such as pneumonia and influenza).

The term “aged” as used herein with reference to a biological sample refers to a sample derived from a subject or donor that is no longer considered young for that species. In one embodiment, the aged biological sample is from an organism about 60 years of age or greater.

The term “allogeneic” as used herein refers to a donor with different histocompatibility complex antigen, i.e., a non-identical donor. In certain embodiments described herein, the umbilical blood cord cells disclosed herein are from an allogeneic source.

The term “autologous” as used herein refers to a genetically identical donor. In certain embodiments, the compositions disclosed herein are not autologous.

The term “biological sample” as used herein refers to tissues, cells or component parts of a whole organism (e.g., body fluids, including but not limited to blood, mucus, lymphatic fluid, synovial fluid, cerebrospinal fluid, saliva, amniotic fluid, amniotic cord blood, placental blood, urine, vaginal fluid and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule. In certain embodiments here, the biological sample used to condition the composition is derived from an adult. In certain embodiments herein, the biological sample is obtained by non-invasive methods. In certain embodiments, the biological sample comprises a multipotent cell, a pluripotent cell or a combination thereof.

The term “biomarker” as used herein refers to a measurable characteristic, the presence of which is indicative of some process (e.g., agent), event or condition (e.g., a disease or disorder). Biomarkers can be classified based on their characteristics (e.g., molecular biomarkers, imaging biomarkers or phenotypic biomarkers) or their use (e.g., diagnostic, monitoring, pharmacodynamic/response, predictive, prognostic, safety, and susceptibility/risk). Representative, non-limiting molecular biomarkers include peptides, proteins, nucleic acids, lipids or the like.

The term “cancer” as used herein encompasses proliferative disorders, neoplasms, precancerous cell disorders and cancers. Thus, a “cancer” refers to any cell that undergoes aberrant cell proliferation that can lead to metastasis or tumor growth.

The term “cell” as used herein refers to the smallest structural and functional unit of an organism, typically microscopic and consisting of cytoplasm and a nucleus enclosed in a membrane. The cells utilized in the methods disclosed herein are generally eukaryotic cells. In some embodiments, the cells are aged or young cells. In certain embodiments, the cells are post-partum cells or extra-embryonic cells.

The term “cell culture medium” or “media” as used herein refers to the components of the environment surrounding cells grown and/or expanded in culture. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. In certain embodiments, the composition disclosed herein comprises only trace amounts of cell culture media.

The term “cell population” as used herein refers to a plurality of cells. The cells may be the same or different.

The term “control” as used herein refers to an alternative subject or sample used in an experiment for comparison purposes. A control can be “positive” or “negative.”

The term “combination” as used herein with respect the administration of the disclosed compositions to a subject in combination with at least one additional therapeutic agent is used to encompass administration that is simultaneous, or sequential administration of the composition and the at least one additional therapeutic agent. In certain embodiments, the at least one additional therapeutic agent is admixed with the composition. In other embodiments, the composition and the at least one additional therapeutic agent are administered in combination but in separate doses.

The term “cytokine” as used herein refers to a soluble protein or peptide which is naturally produced by mammalian cells and which act in vivo as humoral regulators at micro-to picomolar concentrations. Cytokines can, either under normal or pathological conditions, modulate the functional activities of individual cells and tissues. In certain embodiments, the cytokine is a hematopoietic cytokine.

The term “culture” as used herein refers to any growth of cells, organisms, multicellular entities, or tissue in a medium. The term “culturing” refers to any method of achieving such growth and may comprise multiple steps. The term “further culturing” refers to culturing a cell, organism, multicellular entity, or tissue to a certain stage of growth, then using another culturing method to bring said cell, organism, multicellular entity, or tissue to another stage of growth. A “cell culture” refers to a growth of cells in vitro. In such a culture, the cells proliferate, but they do not organize into tissue per se. In embodiments where cells are cultured herein, the culture may involve two three-dimensional culturing conditions.

The term “cytokine” as used herein refers to a class of small intercellular proteins secreted by specific cells to mediate and regulate the immune response, inflammation, and hematopoiesis in the human body. Cytokines include pro-inflammatory cytokines and anti-inflammatory cytokines.

The term “disease” and “disorder” are used herein interchangeably to describe a deviation from the condition regarded as normal or average for members of a species or group (e.g., humans), and which is detrimental to an affected individual under conditions that are not inimical to the majority of individuals of that species or group.

The terms “decrease” or “increase” as used herein refers to any change that results from carrying out the methods disclosed herein, for example, a change in a biomarker and more particularly, a change in gene expression, protein production, amount of a symptom, disease, composition, condition, or activity. A decrease or increase can be any individual, median, or average decrease or increase in a condition, symptom, activity, composition in a statistically significant amount. Thus, the decrease or increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The terms “determining,” “measuring,” “assessing,” and “assaying” are used interchangeably herein and refer to both quantitative and qualitative measurement, and include determining if a characteristic, trait, or feature is present or not. Assessing may be relative or absolute. “Assessing the presence of” includes determining the amount of something present, as well as determining whether it is present or absent.

The term “differentiation” as used herein to processes by which unspecialized cells (such as pluripotent stem cells, or other stem cells), or multipotent or oligopotent cells, for example, acquire specialized structural and/or functional features characteristic of more mature, or fully mature, cells.

The term “donor” as used herein refers to “donor” refers to a human or animal from which one or more cells are isolated prior to administration of the cells, or progeny thereof, into a recipient. The one or more cells may be, for example, a population of stem cells (e.g., HSC's) or MNC. In certain embodiments, the donor providing the population of stem cells is different than the donor of the cells revitalized or the subject of the treatment methods disclosed herein.

The term “expand” as used herein refers to a process by which the number of cells in a cell culture is increased due to cell division. In certain embodiments, the mononuclear cells and/or the stem cells (e.g., HSCs) disclosed herein are expanded prior to obtaining a secretome therefrom. In one embodiment, expanding involves an increase of a cell population (e.g., at least 2-fold) without differentiation accompanying such increase.

The term “effective amount” as used herein refers to an amount sufficient to produce a selected effect, e.g., a therapeutic effect.

The term “enrich,” “isolate” and “purify” as used herein are generally synonymous and refer to the enrichment of a desired component (e.g., extracellular vesicles, such as microvesicles and/or exosomes) relative to unwanted material. The terms do not necessarily mean that the desired component is completely isolated or completely pure. Rather, an increase in weight percentage or relative presence is sufficient.

The term “excipient” as used herein refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The term “exosome” as used herein refers to an extracellular vesicle that is between about 30-150 nM in size. Exosomes are formed from fusion of multivesicular bodies with the plasma membrane. Their lipid membranes are enriched in cholesterol and ceramide, expose phosphatidylserine and contain lipid rafts. They contain conserved proteins, such as CD81, CD63, and tissue/cell type-specific proteins that reflect their cellular source.

In certain embodiments, the compositions disclosed herein comprise exosomes. In other embodiments, the compositions disclosed herein consist essentially of exosomes.

The terms “extracellular vesicle” or “ECV” as used herein refers to a vesicle released by a cell. Structurally, ECVs are composed of a lipid-protein bilayer surround an aqueous core that may contain nucleic acids and soluble proteins The ESC may differ with respect to vesicle size, vesicle density, vesicle lipid bilayer composition, extravesicular proteins or genetic material attached to the vesicles, extravesicular proteins or genetic material floating in surrounding biological sample, vesicle membrane proteins, intra-vesicular proteins, intravesicular genetic material, biochemical alterations of extravesicular or intra-vesicular genetic material. They may range in size from range in size from 30 to 5,000 nm and include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and apoptotic bodies. ECVs are released under physiological conditions, but also upon cellular activation, senescence, and apoptosis. They may contain coding and non-coding genetic material, e.g., mRNA and microRNA. They can be detected in blood, for example, by flow cytometry or nanoparticle tracking analysis. Particular ECVs can be isolated from others on the basis of, for example, size or surface markers. An “EVC” population is a related term used herein to refer to a certain group of ECVs, which may be the same or different.

The term “growth environment” as used herein refers to an environment in which cells will proliferate, differentiate, or mature in vitro. Features of the environment include the medium in which the cells are cultured, any growth factors or differentiation-inducing factors that can be present, and a supporting structure (such as a substrate on a solid surface) if present.

The term “hematopoiesis” as used herein refers to the process of blood cell development and homeostasis. In adults, it occurs in bone marrow and lymphatic tissues.

The term “hematopoietic cell” or “HS” as used herein refers to the cells that constitute the hematopoietic system and include red blood cells, white blood cells (basophils, eosinophils, neutrophils, mast cells, lymphocytes and monocytes) and platelets.

The term “hematopoietic stem cell” or “HSC” as used herein refers to an immature cell that can develop into all types of blood cells, including white blood cells, red blood cells, and platelets. Specifically, HSCs differentiate into common myeloid progenitors (CMPs) and common lymphoid progenitors (CLPs). CMPs produces cells of the erythroid, granulocytic, monocytic, megakaryocytic, and dendritic lineages, whereas CLPs lead to the derivation of T and B lymphocytes, plasma cells, natural killer cells and lymphoid dendritic cells. Terminal differentiation of myeloid lineage cells ultimately leads to the generation and renewal of red blood cells, granulocytes, monocytes, myeloid-derived dendritic cells, and platelets. Hematopoietic stem cells are found, for example, in the bone marrow, peripheral blood and umbilical cord blood (UCB). In a particular embodiment, the compositions and methods of the disclosed herein do not utilize HSCs derived from bone marrow, i.e., derived by invasive methods. Markets for HSCs include Sca-1, CD27, CD34, CD38, CD43, CD48, CD117 and CD150. In a particular embodiment, the compositions and methods disclosed herein are adult HSCs. “HSPC” as used herein refers to both hematopoietic stem cells and hematopoietic stems cells.

The term “immune cells” as used herein refers to B-lymphocytes, T-lymphocytes, NK cells, macrophages, mast cells, monocytes and dendritic cells. In certain embodiments, the cells revitalized using the compositions and methods disclosed herein are immune cells.

The term “in vitro” as used herein refers to an artificial environment and to processes or reactions that occur within an artificial environment. In vitro environments can consist of, but are not limited to, test tubes and cell culture. In contrast, the term “in vivo” refers to the natural environment (e.g., an animal or a cell) and to processes or reaction that occur within a natural environment.

The term “isolated” as used herein with reference to cell refers to cells removed from their original environment.

The term “kit” as used herein refers to any system for delivering the compositions or pharmaceutical compositions disclosed herein. The kit may optionally include one or more enclosers (e.g., one or more containers) and/or supporting materials (e.g., instructions for preparing or using the compositions).

The term “life expectancy” as used herein refers to the mean number of years remaining for an individual subject or group of subjects at a given age.

The term “medium” as used herein refers to an aqueous mixture suitable for cultivation of cells, particularly mammalian cells. The aqueous mixture is typically a solution, although suspensions and colloidal mixtures are also comprised. The medium is typically liquid, although some media can also be temporarily frozen, e.g., for storage purposes.

The term “mesenchymal stem cell” or “MSC” are used herein refers to a type of adult stem cell that has the capacity to self-renew and differentiate into different germs lines such as ectoderm, mesoderm and endoderm. Markets of mesenchymal stem cells include, for example, CD44, CD90, CD105, CD106, CD166, and Stro-1. They are found in/can be isolated from a variety of biological sources, including bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum.

The term “metabolomic” as used herein with respect to studies of cell, tissue or organ revitalization herein refers to the study of cellular metabolites, such as the complete set of metabolites (the metabolome) in a biological sample under a given set of condition. In certain embodiments, the methods disclosed herein result in a change in one or more metabolites in a biological sample. The method may comprise, for example, obtaining a bodily fluid (e.g., urine, blood) or tissue sample from a subject, collecting a metabolic profile from the bodily fluid or tissue sample and comparing the metabolic profile to a reference profile (e.g., a biological sample from the subject prior to carrying out the method). In certain embodiments, the metabolomic protein is obtained by mass Spectrometry (MS) coupled with gas chromatography (GC-MS) or liquid chromatography (LC-MS), high performance liquid chromatography (HPLC), or nuclear magnetic resonance (NMR) spectroscopy.

The term “microRNA” or “miRNA” as used herein refers to class of small, generally 18-to 28-nucleotide-long, noncoding RNA molecules.

The term “microvesicle” or “MV” as used herein refers to an extracellular vesicle shed from a cell, typically in the size range of about of 100 nm to 1 μm. They are formed by outward blebbing of the plasma membrane. Their lipid membrane composition includes exposed phosphatidylserine, enriched in cholesterol and diacylglycerol, contain lipid rafts.

The term “mobilized peripheral blood” refers to peripheral blood treated with a mobilizing agent, or a combination of mobilizing agents. In this context, mobilization refers to the recruitment of stem and progenitor cells from the bone marrow into the blood stream where they can be collected via leukapheresis. The mobilizing agent may be any suitable agent, e.g., a small molecule, a polypeptide, a nucleic acid, a carbohydrate, an antibody, or any other agent that acts to enhance the migration of stem cells from the bone marrow into the peripheral blood. In one embodiment, the mobilization agent is granulocyte colony stimulating factor (G-CSF) (Neupogen®), plerixafor (Mozibil®) or a combination thereof.

The term “mononuclear” as used herein refers to a cell found in blood that has a single, round nucleus. Mononuclear cells may include peripheral blood mononuclear cells (PB-MNCs) or bone marrow mononuclear cells (BN-MNC).

The term “nucleic acid” as used herein refer to a double or single-stranded polymer of ribonucleotide or deoxyribonucleotide bases. A nucleic acid can be recombinant and peptides, e.g., exogenous polypeptides, can be expressed when the nucleic acid is introduced into a cell. Nucleic acids can, for example, include vectors, messenger RNA (mRNA), single stranded RNA that is complementary to an mRNA (antisense RNA), microRNA (mi RNA), tRNA, small interfering RNA (siRNA), small or short hairpin RNA (shRNA), long non-coding RNA (IncRNA), chromosomal DNA, e.g., double stranded DNA (dsDNA), and/or self-replicating plasmids. In certain embodiments, the ECVs disclosed herein contain one or more nucleic acids.

The term “one or more” as used herein includes a number higher than one. For example, two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number, such as plurality or population.

The term “organ” as used herein refers to two or more adjacent layers of tissue, which layers of tissue maintain some form of cell-cell and/or cell-matrix interaction to form a microarchitecture.

The term “parenteral administration” as used herein refers to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

The term “peripheral blood” refers to the circulating blood of the organism. Peripheral blood is composed of erythrocytes, leukocytes and thrombocytes. These blood cells are suspended in blood plasma, through which the blood cells are circulated through the body.

The terms “peripheral blood mononuclear cells” or “PBMCs” refers to a heterogeneous population of blood cells having a round nucleus. Examples of cells that may be found in a population of PBMCs include lymphocytes such as T cells, B cells, NK cells (including natural killer T cells (NKT cells) and cytokine-induced killer cells (CIK cells)) and monocytes such as macrophages and dendritic cells. These cells can be extracted from whole blood or buffy coat samples using a hydrophilic colloid and density gradient centrifugation.

The term “plasma” as used herein refers to the liquid component of cells, for example amniotic cells or blood cells, and may comprise the extracellular matrix of these cells. In some embodiments, the plasma may be derived from blood cells, and may be a human or other plasma. Typical plasma content may include various proteins and other components, for example serum albumins, globulins, fibrinogen, glucose, clotting factors, hormones, electrolytes, and carbon dioxide. Plasma may be generated by any method known in the art, and in certain embodiments is human blood plasma.

The term “pre-primed” are used herein interchangeably to refer to a composition that has been contacted with or exposed to a priming agent(s) prior to administration or use. In certain embodiments disclosed herein, the priming agent is a secretome of an aged MNC. Pre-priming may modulate the composition of the secretome of a given cell (e.g., a HSC cell, or more particularly a UBC cell). In certain embodiments, the methods disclosed herein may involve pre-conditioning the pre-primed composition by one or more methods selected from hypoxic pre-conditioning or small molecule preconditioning (e.g., paclitaxel pre-conditioning).

The term “prevent” as used herein refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “progenitor cell” as used herein refers to a descendant of a stem cell which is capable of further differentiation into one or more kinds of specialized cells, but which cannot divide and reproduce indefinitely. Progenitor cells may be multipotent, oligopotent, or unipotent, and are typically classified according to the types of specialized cells they can differentiate into (e.g., hematopoietic progenitor cells).

The term “proteomics” as used herein refers to the study of the proteome, which includes the all the proteins expressed by a given organism, biological system, tissue or cell at a given time under given conditions. In certain embodiments, the methods disclosed herein may result in the change in one or more aspects of the proteome of a treated cell, tissue or organ.

The term “reactive oxygen species” or “ROS” as used herein refers to highly reactive chemicals formed from O2. Examples of ROS include peroxides, superoxide, hydroxyl radical, singlet oxygen, and alpha-oxygen. In certain embodiments, the methods disclosed herein produce a reduction in ROS or a stabilization in ROS, compared to a control.

The term “rejuvenating” as used herein refers to the decrease in any symptom or parameter associated with aging or alternatively, to the increase in any characteristic or parameter associated with youthfulness.

The term “secretome” as used herein refers the totality of all substances released by a cell (e.g., an aged MNC, a HSC) to the outside of the cell (e.g., to the extracellular space or fluid) It is includes both soluble factors (e.g., growth factors, cytokines, chemokines, and enzymes) and extracellular vesicles (e.g., microvesicles, exosomes, apoptotic bodies). A secretome may be optionally further processed, e.g., to deplete the secretome of one or more components. In other embodiments, one or more components are added to the secretome that are not otherwise naturally present. The composition of the secretome depends species, cell type, isolation procedure and the chemical and physical stimuli to which the cell is exposed.

The term “senescence” as used herein refers to the irreversible growth arrest of a cell that occurs as a result of different damaging stimuli, including DNA damage, telomere shortening and dysfunction or oncogenic stress. Biomarkers of senescence include, for example, senescence-associated β-galactosidase (SA-β-gal) activity.

The term “subject” as used herein refers to animals such as mammals, including, but not limited to, primates (e.g., humans, non-human primates), cows, horses, pigs, sheep, goats, dogs, cats, rabbits, rats, mice and the like. In certain embodiments, the subject is a human.

The term “substantially purified” as used herein with reference to a population of cells means a population of cells substantially homogeneous for a particular marker or combination of markers. By substantially homogeneous is meant at least 90%, and preferably 95% homogeneous for a particular marker or combination of markers. In certain embodiments, the mononuclear cells and/or hematopoietic stem cells disclosed herein are substantially purified.

The term “stem cell” as used herein refers to Stem cells are classified by their developmental potential as totipotent (able to give rise to all embryonic and extraembryonic cell types), pluripotent (able to give rise to all embryonic cell types) or multipotent (e.g., able to give rise to a subset of cell lineages, but all within a particular tissue, organ, or physiological system, such as the hematopoietic system). Representative, hematopoietic stems cells and mesenchymal stem cells. In certain embodiments disclosed herein, the stem cell is an adult stem cell and more particularly, an HSC. In certain embodiments, the stem cell is a placental stem cell.

The term “therapeutic” as used herein means a treatment and/or prophylaxis. A therapeutic effect is obtained by suppression, remission, or eradication of a disease state.

The term “therapeutic agent” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The agent may be provided as is or as a component of a composition. Representative, non-limiting examples of therapeutic agents include small molecules, proteinaceous molecules (e.g., peptides), polypeptides, proteins, genetic molecules (e.g., RNA, DNA and mimetics and chemical analogs thereof) and cellular agents.

The term “tissue” refers to a group or layer of similarly specialized cells which together perform certain special functions.

The terms “treat” or “treating as used herein refers to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a subject, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those with an increased likelihood of infection, those suspected of having cancer, those suspected of harboring an infection, etc.).

The term “umbilical cord blood” or “UBC” as used herein refers to blood that remains in the umbilical cord and placenta after childbirth. UBC contains multiple populations of stem/progenitor cells, capable of giving rise to hematopoietic, epithelial, endothelial, and neural tissues both in vitro and in vivo. UBC can be obtained commercially, e.g., LifeBank Inc., ViaCord, or other sources.

The term “umbilical cord blood cell” as used herein refers to hematopoietic stem cell or progenitor cell found in UCB.

The term “young” as used herein is used to refer to a subject that is of chronological age of 40 years old or younger, e.g., 35 years old or younger, including 30 years old or younger, e.g., 25 years old or younger or 22 years old or younger. As such, “young” may refer to a subject that is between the ages of 0 and 40, e.g., 0, 1, 5, 10, 15, 20, 25, 30, 35, or 40 years old. In other instances, “young” and “young individual” may refer to a biological (as opposed to chronological) age such as an individual who has not exhibited the levels of inflammatory cytokines in the plasma exhibited in comparatively older individuals. Conversely, these “young” individuals may refer to a biological (as opposed to chronological) age. In contrast, certain cells disclosed herein are “aged”.

### Compositions

Disclosed herein are acellular, allogenic compositions comprising at least one extracellular vesicle (ECV) derived from a pre-primed biological sample comprising a population of HSCs (e.g., UCB cells). These compositions may be, for example, pre-primed secretome compositions derived from UBC cells.

All cells, prokaryotes and eukaryotes, release ECVs as part of their normal physiology and during acquired abnormalities. ECVs can be classified as ectosomes or exosomes, depending on their origination. Ectosomes, which include microvesicles, microparticles and large vesicles), are vesicles that pinch off the surface of the plasma membrane via outward budding. Exosomes, in contrast, have an endosomal origin.

In one embodiment, the composition comprises a plurality of ECVs derived from a pre-primed biological sample. The ECVs may be the same or different. In one embodiment, the ECVs include ectosomes (e.g., microvesicles), exosomes or a combination thereof.

In one embodiment, the unit dose of the composition disclosed herein includes about one million ECVs or more, more particularly, about one million, about two million, about three million, about four million or about five million ECVs or more. In a particular embodiment, the unit dose of the composition is about 200 microliters or more, more particularly, about 250 microliters, about 300 microliters, about 350 microliters, about 400 microliters, about 450 microliters or about 500 microliters or more.

The size of the ECVs may vary. In certain embodiments, the ECVs are between about 1 and about 1000 nm. In one embodiment, the compositions comprises a plurality of ECVs less than about 200 nm, less than about 400 nm, less than about 600 nn, less than about 800 nm, or less than about 1000 nm. In a particular embodiment, the composition excludes ECVs greater than about 1 pm.

In one embodiment, the composition comprises at least one microvesicle (MV), exosome or combination thereof.

In a particular embodiment, the composition consists essentially of a plurality of MVs.

In a particular embodiment, the composition consists essentially of a plurality of exosomes.

In a particular embodiment, the composition consists essentially of a plurality of MVs and exosomes.

In certain embodiments, the ECVs may be chemically or biologically modified. For example, the ECVs may be enriched for one or more RNA species (mRNA, microRNA, tRNA) or proteins of interest.

The source of the ECVs may vary. In one embodiment, the ECV source is a stem cell (i.e., stem cell-derived ECVs) and more particularly, an adult stem cell. An adult stem cell is generally a multipotent undifferentiated cell found in tissue comprising multiple differentiated cell types. The adult stem cell can renew itself and, under normal circumstances, differentiate to yield the specialized cell types of the tissue from which it originated, and possibly other tissue types.

Representative, non-limiting stem cells include hematopoietic stem cells (HSC), mammary stem cells, intestinal stem cells, mesenchymal stem cells, endothelial stem cells, neural stem cells, olfactory adult stem cells, neural crest stem cells, and testicular cells. Sources for adult stem cells include, for example, bone marrow, adipose tissue, peripheral blood (PB), umbilical cord blood or placental blood).

In one embodiment, the stem cell is derived from extra-embryonic tissue, i.e., tissue associated with, but not originating from, the embryo or fetus. Extra-embryonic tissues include extraembryonic membranes (e.g., chorion, amnion, yolk sac and allantois), umbilical cord, and placenta.

In some embodiments, the cells have been cultured. Culture procedures that support the adult HSCs are known in the art. See, e.g., Baron F, et al., Exp Rev Hematol. 2016;9(3):297-314.Culture procedures that support MNCs are known in the art. J. Bara et al., Cytotherapy, 2015 April; 17(4):458-72. In a particular embodiment, aged TNC and UCB cells are first purified from a donor sample and store in liquid nitrogen. Thereafter, the cells are cultured.

In a particular embodiment, the stem cell is an adult HSC. HSCs are characterized by their capacity for self-renewal and pluripotency (Orkin et al., Cell 132:631-644 (2008). HSCs can be found in can be found in, for example, peripheral blood (PB), bone marrow (BM), and umbilical cord blood (UCB). Specifically, HSCs can differentiate into all types of blood cells, including myeloid-lineage and lymphoid-lineage cells. Myeloid-lineage cells include all blood cells except lymphoid cells. The lymphoid-lineage cells consist of T, B, and natural killer (NK) cells, which are relevant to innate and adaptive immune cells. From HSC to mature cells, there are several intermediate progenitor cells. HSC can be divided into LT-HSC, ST-HSC, and multipotent progenitor (MPP) in terms of duration of repopulation. Aging is relevant to the functional decline of normal HSCs.

In a particular embodiment, the ECV may be derived from HSCs or hematopoietic progenitor cells (HPCS) present in umbilical cord blood (UCB). UCB is a clinically useful, non-invasive source of such cells. Moreover, HSCs and HPCs derived from umbilical cord blood do not trigger significant immune responses, permitting universal or near-universal use. In one embodiment, the HSCs are isolated from are isolated from at least about 60%, about 70%, about 80%, 90% or 95% or more of the constituents found naturally in UCB.

In certain embodiments, the ECV may be derived from the secretome of HSCs from more than one donor (e.g., more than one umbilical cord).

In one embodiment, the ECVs are derived from a pre-primed biological sample, e.g., a biological sample comprising HSCs contacted with a priming agent(s). Optionally, the biological sample is depleted of mature cells, e.g., T-cells.

The priming agent may vary. In one embodiment, the priming agent is a aged secretome of a aged mononuclear cell (MNC). Mononuclear cells represent the enriched lymphocyte and monocyte fraction of whole blood. They may be isolated from adult peripheral blood cells, for example. They may also be isolated from adult plasma or adult bone marrow.

Studies have shown that PB-MNCs differ between aged and young individuals. Specifically, PB-MNCs secrete more inflammatory mediators (e.g., TNF-α and IL-6) and anti-inflammatory cytokines (e.g., IL-10).

The aged secretome may be a total secretome or isolate thereof.

In certain embodiments, the composition disclosed herein may comprise one or more additional components in addition to the one or more ECVs. Representative, non-limiting components include proteins, peptides, proteinaceous molecules, nucleic acids, lipids, hormones or a combination thereof.

In a particular embodiment, the composition may comprise one or more cytokines. The cytokines may be, e.g., hematopoietic cytokines. Representative, non-limiting cytokines include erythropoietin (EPO), granulocyte-colony stimulating factor (G-CSF) granulocyte-macrophage colony stimulating factor (GM-CSF), interleukins (e.g., IL2, IL-3, IL-5, IL-6, IL-7, IL-11), macrophage-colony stimulating factor (M-CSF), stem cell factor (SCF), thrombopoietin (TPO) or a combination thereof.

In one embodiment, the composition is provided in the form of the total secretome of a pre-primed biological sample (e.g., a biological sample comprising HSCs and more particularly, UCB cells).

In alternate embodiments, the composition is provided in the form of an isolate or fraction of the total secretome (e.g., an isolate enriched for ECVs, or more particularly, enriched for MVs, exosomes or a combination thereof).

In a particular embodiment, the total secretome is depleted of a significant percentage of soluble secreted proteins, e.g., about 60%, about 70%, about 80%, about 90%, about 100%.

In another particular embodiment, the total secretome is depleted of a significant percentage of apoptotic bodies, e.g., about 60%, about 70%, about 80%, about 90%, about 100%.

In one embodiment, the composition comprises the ECV fraction of the total secretome of a pre-primed biological sample (e.g., a biological sample comprising HSCs and more particularly, UCB cells).

In one embodiment, the composition comprises the MV fraction of the total secretome of a pre-primed biological sample (e.g., a biological sample comprising HSCs and more particularly, UCB cells).

In one embodiment, the composition comprises the exosome fraction of the of the total secretome of a pre-primed biological sample ((e.g., a biological sample comprising HSCs and more particularly, UCB cells).

In one embodiment, the composition comprises the combined MV fraction and exosome fraction of the total secretome of a pre-primed biological sample (e.g., a biological sample comprising HSCs and more particularly, UCB cells).

In one embodiment, the composition consists essentially of ECV fraction of the total secretome of a pre-primed biological sample (e.g., a biological sample comprising HSCs and more particularly, UCB cells).

In one embodiment, the composition consists essentially of the MV fraction of the of the total secretome of a pre-primed biological sample (e.g., a biological sample comprising HSCs and more particularly, UCB cells).

In one embodiment, the composition consists essentially of the exosome fraction of the of the total secretome of a pre-primed biological sample (e.g., a biological sample comprising HSCs and more particularly, UCB cells).

In one embodiment, the composition consists essentially of the combined MV and exosome fraction of the total secretome of a pre-primed biological sample (e.g., a biological sample comprising HSCs and more particularly, UCB cells).

In certain embodiments, the disclosed composition is a pharmaceutical composition, i.e., includes the composition disclosed herein and at least one pharmaceutically acceptable carrier, diluent, vehicle or excipient, depending upon the route of administration and the preparation desired. The phrase “pharmaceutically acceptable carrier” refers to a carrier or a diluent that does not cause significant irritation to an organism and does not abrogate the biological activity and properties of the administered microvesicles or secretome. Methods of formulating suitable pharmaceutical compositions are known in the art, see, e.g., Remington: The Science and Practice of Pharmacy, 21st ed., 2005; and the books in the series Drugs and the Pharmaceutical Sciences: A Series of Textbooks and Monographs (Dekker, NY).

To formulate a pharmaceutical composition, the composition (e.g., total secretome, ECV-enriched secretome, plurality of ECVs (e.g., microsomes, exosomes, or a combination thereof)) may be dissolved, suspended or dispersed in a pharmaceutically accepted carrier, diluent or vehicle in an effective amount and concentration.

The compositions disclosed herein may be provided as a dosage form. Representative, non-limiting dosage forms include liquid dispersions, gels, aerosols, lyophilized formulations, tablets, or capsules.

In a particular embodiment, the composition is provided in an injectable form. Injectable formulations can be prepared in conventional forms, such as emulsions, microemulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof. The suspensions can be lyophilized.

The pharmaceutical compositions disclosed herein may be co-formulated with other active ingredients which do not impair the desired therapeutic action, or with substances that supplement the desired action.

The compositions (e.g., pharmaceutical compositions) disclosed herein are desirably stable under conventional storing conditions. For example, in certain embodiments, the compositions exhibit stability at 4° C., 20° C. or 25° C. for at least 5 days, at least 7 days, at least two weeks or at least one month or more.

In certain embodiments, the composition (e.g., pharmaceutical composition) is lyophilized and then reconstituted with an appropriate diluent at the time of use or administration.

### Methods of Use

Also disclosed herein are methods of using the compositions (e.g., pharmaceutical compositions) disclosed herein, including but not limited to therapeutic methods of use.

In one embodiment, a method for using the composition is provided comprising (i) contacting the composition with a cell, tissue or organ.

In a particular embodiment, the contacting occurs in vitro. According to this embodiment, the contacting may occur, for example, in a test tube, culture dish, or elsewhere outside a living subject.

In certain embodiments, the method comprises contacting the contacting the composition with aged human bone marrow.

In a particular embodiment, the contacting occurs in vivo. According to this embodiment, the contacting occurs inside of a living subject, such as a human or non-human (animal) subject.

In a particular embodiment, the non-human animal is a yeast, roundworm, fruit fly, rodent (e.g., rat or mouse), bird or non-human primate.

In a particular embodiment, the non-human animal is a mouse. Similarities and differences between mouse and man in relation to studies on ageing have been reviewed. See, e.g., Vanhooren, V. et al., Ageing Res. Rev., 12 (2013), pp. 8-21 (incorporated herein by reference).

In a particular embodiment, the animal subject is a humanized mouse. See, e.g., Shultz L D, et al. al. Nat Rev Immunol 2007; 7:118-130.

In another embodiment, a method of rejuvenating a cell, tissue and/or organ is disclosed, comprising (i) contacting a cell, tissue and/or organ with an effective amount of the composition, thereby rejuvenating the cell, tissue and/or organ.

In a particular embodiment, the contacting occurs in vitro. According to this embodiment, the contacting may occur, for example, in a test tube, culture dish, or elsewhere outside a living subject.

In a particular embodiment, the contacting occurs in vivo. According to this embodiment, the contacting occurs inside of a living subject, such as a human or non-human (animal) subject. In certain embodiments, the subject is a humanized mouse

In a particular embodiment, the cell is an immune cell or more particularly, a T-cell.

In certain embodiment, the method further comprises (i) determining the rejuvenation of the cell, tissue or organ. In this context, a rejuvenated cell is one that has one or more characteristics of a younger cell (e.g., gene expression, methylation profile, activity profile, phenotypical profile, mitochondrial function and health, cytokine expression or the like) while retaining one or more cell identity markers.

Representative, non-limiting methods for determining the rejuvenation include conducting (i) mitogen and antigen stimulation studies to assess B cell and T cells activity, (ii) cytotoxic activity assays to assess natural killer cell activity, (iii) long-term culture-initiating cell and cobblestone area-forming cell assays to assess stem and progenitor cells, and (iv) assessing clonal hematopoiesis of indeterminate potential to assess the relative age and functional age of the immune cells.

In a particular embodiment, the method comprises determining rejuvenation utilizing an LT-HSC/Cobblestone Assay.

In one embodiment, the method for determining the rejuvenation of the cell comprises long-term culture-initiating cell-forming cell assays to assess stem and progenitor cells.

In a particular embodiment, the method comprises determining rejuvenation by identifying a change in one or more biomarkers association with aging. In a particular embodiment, the method comprises determining rejuvenation by identifying a change in one or more biomarkers association with lymphoid/myeloid bias. In a particular embodiment, the method comprises determining rejuvenation by identifying a change in one or more markers associated with hematopoietic proliferative potential. Examples of aged-associated markers include: CD41 (integrin associated with aging), Selectin P (SELP), & Clusterin (Clu).

Assessment of revitalization will also be performed by assessing degree of inflammation by measuring the expression of inflammatory cytokines. Non-limiting examples of inflammatory cytokines include interleukin-1 (IL-1), IL-12, and IL-18, tumor necrosis factor alpha (TNF-α), interferon gamma (IFNγ), and granulocyte-macrophage colony stimulating factor (GM-CSF).[3]

Assessment of revitalization may also comprise ChiP single-cell sequencing to ensure that aged clones are no longer proliferating.

In one embodiment, the method results in the rejuvenation of a cell compared to a control (i.e., a cell not contacted with the composition) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the method results in the rejuvenation of a tissue compared to a control (i.e., a tissue not contacted with the composition) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the method results in the rejuvenation of a tissue compared to a control (i.e., a tissue not contacted with the composition) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The compositions disclosed herein are also suitable for use in preventing aging of a cell, tissue or organ. Aging is characterized by structural and functional changes at the molecular, cellular, tissue and organismal levels.

In vitro assays for assessing cell aging are known in the art.

In another embodiment, a method of treating or preventing aging is provided comprising administering a therapeutically effective amount of the composition to a subject in need thereof, thereby treating or preventing aging. Prior to administering the compounds or compositions, the subject can be diagnosed with a need for treatment of age-related disorder or disease,

In a particular embodiment, aging is delayed or reduced in the subject.

In certain embodiments, aging is delayed or reduced over a defined time period as recognized by a medically recognized technique. For example, aging may be delayed or reduced about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to a control group or with respect to predicted age, e.g., AI-driven age prediction.

The delay in aging may be measured with respect to a biomarker. The biomarker may be a molecule-based, blood based, physiological, neurological or an AI-driven age predictor. See Jia, L., et al. (2017). Clin. Interv. Aging 12, 759-772 (incorporated herein by reference). In one embodiment, the delay in aging is measured with respect to (i) a primary hallmark of aging (e.g., genomic instability, telomere attrition, epigenetic alteration, and loss of (ii) an antagonistic hallmark of aging (e.g., deregulated nutrient sensing, mitochondrial dysfunction, and cellular senescence; or (iii) an integrative hallmark of aging (e.g., stem cell exhaustion and altered intercellular communication). See Longo, V. et al. (015). Aging Cell 14, 497-5.

In one embodiment, the delay in aging is measured with respect to a physiological characteristic. The physiological trait may vary and include, for example, blood pressure, heart rate variability, pulse waive velocity, gait speed, grip strength or a combination thereof.

In a particular embodiment, the delay in aging is measured with respect to a phenotypic trait. In one example, the phenotypic trait is frailty, which can be measured with respect to five deficits: weight loss, exhaustion, muscle weakness, slow walking speed, and low physical activity.

In one embodiment, the delay in aging is measured by a blood-based biomarker, e.g., fasting insulin, hemoglobin A1C or insulin-like growth factor 1 or the like.

In a particular embodiment, aging is delayed about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to a control group.

Several immune deficits have been described in aged subjects. See, e.g., Pawelec G. et al., Exp Gerontol 2018; 105:4-9. In particular, age may impact the functions of lymphocytes, especially T lymphocytes. These aging-related changes may vary by cell.

For example, aged macrophages and neutrophils have impaired respiratory burst and reactive nitrogen intermediates as a result of altered intracellular signaling, rendering them less able to destroy bacteria. Aged neutrophils are also less able to respond to rescue from apoptosis. Aged dendritic cells (DC) are less able to stimulate T and B cells. For example, aged T-cells and B-cells may exhibit reduced proliferation or stimulation and/or changes in the prevalence of particular subsets of such cells. For example, NK cells may be less capable of killing tumor cells. They may also have increased interleukin-4 production.

As a result, aged subjects may experience poorer responses to vaccination, lower capacity to mediate anti-cancer responses, more inflammation and tissue damage, along with autoimmunity and loss of control of persistent infections.

In one embodiment, a method of improving the immune response in a subject in need thereof of (e.g., an aged subject) is provided comprising administering a therapeutically effective amount of the composition to the subject, thereby improving the subject's immune response.

In certain embodiments, the method may result in increased T cell proliferation or increased B cell proliferation.

In a particular embodiment, the immune response is improved about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to a control group.

Increased inflammation has been described in aging subjects. In particular, aging has been associated with chronic low-grade increases in the circulating levels of inflammatory mediators including tumor necrosis factor-alpha (TNF-α), interleukin-6 (IL-6), and interleukin-1 alpha (IL-1β; Brüünsgaard and Pedersen

In one embodiment, a method of reducing inflammatory cytokines in a subject in need thereof of (e.g., an aged subject) is provided comprising administering a therapeutically effective amount of the composition to the subject, thereby reducing the presence of inflammatory cyokines.

In a particular embodiment, inflammatory cytokines are reduced about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to a control group.

In a further embodiment, a method of treating or preventing an age-related disease or disorder is provided, comprising an therapeutically effective amount of the composition to a subject in need thereof, thereby treating or preventing the disease or disorder.

In a particular embodiment, the method results in the slowing of the progression or onset of the age-related disease or disorder over a defined time period as measured by a medically recognized technique. For example, the progression or onset of the disease or disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group or AI-drive disease progression predictor.

In one embodiment, the slowing of the progression of onset of a disease or disorder is measured with respect the presence of a biomarker(s). In certain embodiments, the slowing or progression of the disease or disorder can be measured with respect to the physical, social, emotional, and cognitive functioning, pain, vitality, and over-all wellbeing of the subject.

Physical functioning may be assessed with respect to physical mobility and independence. Common aspects measured include fitness or physiologic health by clinical or objective measures, basic self-care activities (activities of daily living, ADLs), or more complex self-care activities (instrumental ADLs, IADLs).

In one embodiment, the methods disclosed herein result in an slowing in the decline of physical functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

In another embodiment, the methods disclosed herein result in an improvement in physical functioning of about bout 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

Social functioning measures often include social role functioning, community involvement, quality of interpersonal relationships, and coping capacity.

In one embodiment, the methods disclosed herein result in an slowing in the decline of social functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

In another embodiment, the methods disclosed herein result in an improvement in social functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

Emotional functioning describes the range of affective well-being, specifically positive and negative emotions and emotional stability.

In one embodiment, the methods disclosed herein result in an slowing in the decline of emotional functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group, utilizing a medically-recognized tools.

In another embodiment, the methods disclosed herein result in an improvement in emotional functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group, using a medically-recognized tool.

Cognitive functioning measures include memory, reasoning, and orientation to describe range of intellectual ability.

In one embodiment, the methods disclosed herein result in an slowing in the decline of cognitive functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

In another embodiment, the methods disclosed herein result in an improvement in cognitive functioning of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% with respect to a control group.

The disease or disorder may be any suitable disease or disorder. In one embodiment, the disease or disorder is aging-related.

The aging-related disorder may be any aging-related disorder. Representative, non-limiting examples of aging-related disorders include Alzheimer's disease, aneurysm, arthritis, atherosclerosis, cystic fibrosis, fibrosis in pancreatitis, glaucoma, hypertension, idiopathic pulmonary fibrosis, inflammatory bowel disease, intervertebral disc degeneration, degenerative joint disease, macular degeneration, osteoarthritis, type 2 diabetes mellitus, obesity, adipose atrophy, lipodystrophy, atherosclerosis, cataracts, COPD, idiopathic pulmonary fibrosis, urinary incontinence, kidney transplant failure, prostatic hyperplasia, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, ocular neovascularization, cachexia, glomerulosclerosis, fatty liver disease, liver cirrhosis, NAFLD, osteoporosis, amyotrophic lateral sclerosis, Huntington's disease, spinocerebellar ataxia, multiple sclerosis, neurodegeneration, stroke, cancer, dementia, vascular disease, infection susceptibility, chronic inflammation, and renal dysfunction.

In a particular embodiment, the aging-related disease or disorder is cancer, cardiovascular disease, diabetes, stroke, neurodegeneration, chronic lung disease, cognitive impairment, arthritis, osteoporosis, or vision loss.

In one embodiment, the aging-related disorder is cancer.

Representative, non-limiting cancers that can be treated or prevented utilizing the methods of the present invention include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In some embodiments, the cancer is selected from the group consisting of melanoma, colorectal cancer, pancreatic cancer, bladder cancer, breast cancer, triple negative breast cancer, ovarian cancer and lung cancer.

In one embodiment, slowing of the progression or onset of the cancer may be measured with respect to a biomarker, such as a molecule-based, radiographic-based, histologic-based or physiological biomarker.

In a particular embodiment, the biomarker is selected from: ALK, AKT1, AKT2, AKT3, BRAC1, BRAC2, BRAF, FGFR, FGFR1, FGFR2, HER2, JAK1, JAK2, KRAS, MDM2, MET, NTRK1, NTRK2, NTRK3, NRG, PTEN, PI3KCA, RAD51B, RAD51C, RAD51D, and RAD54L.

In another embodiment, the aging-related disorder is cardiovascular disease.

Representative, non-limiting cardiovascular disease or disorders that can be treated or prevented using the methods of the present invention include diseases or disorders of the heart, heart valves, and/or blood vessels and their subcomponents (e.g., vascular/venous valves). For example, cardiovascular disease or disorders include angina pectoris, pre-infarction angina, myocardial infarction, heart failure, ischemia, stroke, acute coronary disease, heart failure, acute heart failure, chronic heart failure, iatrogenic heart disease, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

In one embodiment, slowing of the progression or onset of the cancer may be measured with respect to a biomarker. Representative, non-limiting biomarkers include LDL cholesterol, high-sensitivity C-reactive protein (hsCRP), troponin, a natriuretic peptide (BNP and NT-proBNP) or a combination thereof.

In a further embodiment, the aging-related disorder is a neurological or cognition disorder. For example, the disease or disorder is Huntington's disease, Parkinson's disease, frontotemporal dementia, dementia, Alzheimer's disease, amyotrophic lateral sclerosis, spinal cord trauma, stroke, diffuse traumatic brain injury, HIV-associated dementia, epilepsy, Rett syndrome, dyskinesia, unspecified dystonia, or pseudobulbar affect.

In one embodiment, slowing of the progression or onset of the neurological disorder ay be measured with respect to a biomarker. Representative, non-limiting biomarkers include isoprostanes, tau, Aβ, sulphatides, homocysteine or combinations thereof.

The slowing of the progression or onset of cognitive impairment may be measured, for example, by the Mini-Mental State Examination (MMSE).

In a further embodiment, the aging-related disorder is arthritis. Representative, non-limiting forms of arthritis that can be treated or prevented utilizing the methods disclosed herein include osteoarthritis and rheumatoid arthritis.

The slowing of the progression or onset of osteoarthritis may be measured, for example, by a biomarker, e.g., a biomarker selected from urinary C-terminal telopeptide of collagen type II (CTX-II), urinary Glc-Gal-Pyd and serum N-propeptide II of type II collagen (PIINP) or a combination thereof.

In certain embodiments, the disease or disorder is not age-related but rather an infectious disease or disorder. The infection may be an acute infection, characterized by a rapid onset of illness, a relatively short period of symptoms, and resolution within days, or a chronic infection. is an infection that develops slowly and lasts a long time. The infectious agent may be, for example, a bacterium, a virus, a fungi or prion.

In a particular embodiment, the infectious disease is bacterial or viral pneumonia.

In another embodiment, the method comprising administering a composition disclosed herein to a subject suffering from an injury, for example, a traumatic injury, where administration of the composition results in treating the traumatic injury.

In one embodiment, the method is disclosed for treating or preventing an autoimmune or inflammatory disease or disorder a subject in need thereof, comprises administering a composition disclosed herein to a subject suffering from or at risk of suffering from an inflammatory disease or disorder, wherein administration of the composition results in treatment or prevention of the autoimmune or inflammatory disease or disorder.

The inflammatory disease or disorder may be any suitable inflammatory disease or disorder. Representative, non-limiting inflammatory disease diseases or disorders include rheumatoid arthritis, inflammatory bowel disease (IBS), multiple sclerosis (MS), lupus erythematosus, lupus nephritis, diabetic nephropathy, Alzheimer's disease, Parkinson's disease, amyotrophic lateral sclerosis, Crohn's disease, multiple sclerosis, Guillain-Barre syndrome, psoriasis, Grave's disease, ulcerative colitis, and non-alcoholic steatohepatitis.

In one embodiment, a method is disclosed for decreasing at least one symptom or associated with aging or a disease or disorder (e.g., an age-related disease or disorder) in a subject in need thereof, comprising administering an effective amount of the composition disclosed herein to the subject, thereby decreasing at least one symptom or parameter associated with aging in the subject. The decrease in symptoms may be measured subjectively or objectively. In certain embodiments, the decrease in symptoms is measured for one subject for a group of subjects/

The symptom may be any relevant symptom.

In certain embodiments, the method disclosed herein decreases at least one symptom by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a recognized technique. The decrease may be measured in comparison to a control group, e.g., an untreated group.

The symptom may be any symptom associated with aging. Representative, non-limiting symptoms include pain. Pain often is defined as the degree of debilitating physical discomfort as a sensation, not as a degree of physical functioning. Its measures often capture intensity, frequency, and duration. In a particular embodiment, the method disclosed herein is useful for treating chronic pain (e.g., chronic inflammatory pain). Chronic pain includes any pain requiring treatment for a period of greater than 1 month, for example, 6 months, 8 months, 10 months, 1 year or longer.

In one embodiment, the methods disclosed herein result in a slowing in the decrease in pain of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The decrease may be measured in comparison to a control group, e.g., an untreated group.

In a particular embodiment, the method disclosed herein is useful for treating nociceptive pain, e.g., either somatic or visceral nociceptive pain.

In a particular embodiment, the method disclosed herein is useful for treating neuropathic pain. In certain embodiments, the neuropathic pain may be associated with cancer, an autoimmune disorder, diabetes, a central nervous system disorder or a viral infection.

Methods for determining and scoring levels of pain are known in the art, and include increases in mobility and function, and decreases in allodynia, thermal sensitivity, and self-reported pain on available scaling and/or scoring measures.

The subject treated with the composition may be any suitable subject. The subject may be, for example, an adult, a youth or a child.

In certain embodiments, the subject is an adult. In a particular embodiment, the subject more than about 50% through its expected lifespan, such as more than 60%, e.g., more than 70%, such as more than 75%, 80%, 85%, 90%, 95% or even 99% through its expected lifespan.

In a particular embodiment, the subject is a human and is about 40, about 45, about 50, about 55, about 60, about 65, about 70, about 75, about 80, about 85 or about 90 years or older.

In a particular embodiment, the subject between about 40 and about 70 years old, more particularly, about 45 and about 65 years old, more particularly, about 50 and about 60 years old.

Any suitable form of administration may be utilized in the methods of disclosed herein. Representative, non-limiting forms of administration include parenteral, e.g., intravenous, intradermal, subcutaneous, oral (e.g., inhalation), transdermal (topical), transmucosal, and rectal administration.

In a particular embodiment, the administration is subcutaneous.

The exact amount of the compositions required will vary from subject to subject, depending on the species, age, weight and general condition of the subject, the mode of administration and the like.

In a particular embodiment, the composition is administered subcutaneously in about 0.5 mL volume or less. In certain embodiment, the composition is administered subcutaneously in about 0.4 mL volume or less, 0.3 mL volume or less, 0.2 mL volume or less, or 0.1 mL volume or less. In certain embodiment, the composition comprises about 1 million ECVs or more, about 2 million ECVs or more, about 3 million ECVs or more, about 4 million ECVs or more, or about 5 million ECVs or more.

In a particular embodiment, the composition is administered in about 200 microliters or more, more particularly, about 250 microliters, about 300 microliters, about 350 microliters, about 400 microliters, about 450 microliters or about 500 microliters or more. In certain embodiment, the composition comprises about 1 million ECVs or more, about 2 million ECVs or more, about 3 million ECVs or more, about 4 million ECVs or more, or about 5 million ECVs or more.

Optionally, the methods disclosed herein may further comprise administering one or more additional therapeutic agents to the subject.

The one or more additional therapeutic agent may be selected from an anti-aging agent, a anti-cancer agent, an anti-cancer agent, an anti-inflammatory agent, an anti-convulsant agent, an anti-microbial agent, an anti-viral agent, an anti-Parkinson agent (e.g., dopamine agonist, precursor), a cardiovascular agent (e.g., a beta-blocker, a calcium channel, vasodilator, atiarrhythmic, diuretic)

In one embodiment, the composition is administered in combination with at least one additional therapeutic agent to achieve an additive or synergistic effect. The term “in combination” as used herein includes (a) concomitantly; (b) as an admixture; (c) separately and simultaneously or concurrently; or (d) separately and sequentially.

The one or more additional therapeutic agent may be any suitable therapeutic agent. Representative, non-limiting therapeutic agents include

Optionally, the administration may occur hourly, daily, weekly, monthly or over some other interval of time. In one embodiment, the administration is annually or bi-annually.

In a particular embodiment, the composition is self-administered.

### Methods of Manufacture

Disclosed herein is a method for making the composition disclosed herein comprising (i) providing an first biological sample (e.g., a biological sample from a subject considered to be an adult for the relevant species) comprising the aged secretome of population of aged mononuclear cells; (ii) providing a second biological sample comprising a hematopoietic stem cell population (HSC) (e.g., UBC cells); (iii) bringing the aged biological sample and the biologic sample into contact for a period of time; (iv) permitting the HSCs to shed a population of extracellular vesicles (e.g., in the extracellular space or culture medium); and (v) collecting the population of extracellular vesicles.

The first biological sample may be adult peripheral blood, adult plasma or adult bone marrow.

The aged secretome may be a total secretome, a secretome depleted of one or more undesirable components or a secretome enriched for one or more desirable components.

In one embodiment, the aged biological sample and the biological sample are brought into contact for a time period less than about 50 hours, more particularly, less than about 48 hours, less than about 46 hours, less than about 44 hours, less than about 42 hours, less than about 40 hours or less than about 38 hours, but in each case greater than one hour.

In a particular embodiment, the aged biological sample and the biological sample are brought into contact for a time period between about 36 and 48 hours, more particularly, about 36, about 38, about 40, about 42, about 44, about 46 or about 48 hours.

The biological sample comprising the HSCs may be any suitable biological sample. Examples include bone marrow (fractionated or unfractioned) or blood (e.g., umbilical cord blood, placental blood, peripheral blood). In certain embodiments, the biological sample is an extra-embryonic sample. In certain embodiments, the biological sample is not embryonic tissue or fetal tissue.

In a particular embodiment, the biological sample is umbilical cord blood (UCB). UCB is blood remaining in the placenta and umbilical cord after the fetus is delivered and the umbilical cord is ligated and dissected. Both the placenta and umbilical cord are typically discarded. In certain embodiments, the UCB is depleted of mature hematopoietic cells such as T cells, B cells, NK cells, dendritic cells, monocytes, granulocytes, erythroid cells, and their committed precursors. Methods of depleting such cells are known in the art, e.g., by immune-depletion.

The aged biological sample may be any suitable sample. Examples include aged peripheral blood, aged plasma aged bone marrow or aged mobilized peripheral blood. In certain embodiments, the aged biological sample is a secretome derived from aged peripheral blood, aged bone marrow or aged mobilized peripheral blood.

The population of ECVs may be present in, for example, cell culture or a bodily fluid (e.g., blood). The resulting composition may be enriched for one or more components or depleted of one or more components.

In certain embodiments, the ECVs are present in cell culture, e.g., from culturing cells within the biological sample. In a particular embodiment, aged-TNC and UBC are first purified from a donor sample and stored in liquid nitrogen. The Aged-TNCs & UCB can then be used at any time for cell culture to produce the pre-primed product.

In one embodiment, the Aged-TNCs are allowed to equilibrate for 24 hours and the UCB cells equilibrate for 12 hours. Following equilibration, the culture dishes containing the aged-TNCs are spun down to remove the cells themselves. The residual secretome (containing the ECVs, e.g., microvesicles, exosomes) are then administered to the UCB cells during the pre-priming phase. The time period of the pre-priming phase may vary. In one embodiment, the pre-priming phase is between about one and about four days, more particularly, about one and about three days or about one and about two days. In certain embodiments, the pre-priming phase is between about 36 and about 48 hours. The pre-primed ECVs (e.g., microvesicles, exosomes) can then be isolated and stored as the final product.

Methods of isolating ECVs, including from different sources, are known in the art. See, e.g., J Clin Invest. 2016; 126:1152-1162; Ling, H. et al., Nat Rev Drug Discov. 2013; 12:847-865. Welton J L, et al., J Extracell Vesicles. 2015;4:27269.

Representative, non-limiting methods of purifying EVCs or subsets thereof include, for example, DC, density-gradient centrifugation (DGC), sucrose cushion centrifugation, gel-permeation chromatography (GPC), affinity capture (AC), microfluidic devices, synthetic polymer-based precipitation, and membrane filtration.

In one embodiment, ECVs are isolated by differential centrifugation (DC), depending on size. After removal of cells in a initial low-speed centrifugation step, ECVS are then isolated at using centrifugal accelerations between about 19,000 and 100,000. Thery C, et al. Curr Protoc Cell Biol. 2006; Chapter 3: Unit 3.22.

Purity of the resulting composition can be assed by any suitable methods. Methods for assessing the purity of ECV compositions include, for example, dynamic light scattering (DLS), nanoparticle tracking analysis (NTA) and electron microscopy.

In a particular embodiment, the ECV component has a purity of greater than about 85%, greater than about 90%, greater than about 93%, greater than about 95%, greater than about 95% or about 99% or greater.

In a particular embodiment, the MV component has a purity greater than about 85%, greater than about 90%, greater than about 93%, greater than about 95%, greater than about 95% or about 99% or greater.

In a particular embodiment, the exosome component has a purity greater than about 85%, greater than about 90%, greater than about 93%, greater than about 95%, greater than about 95% or about 99% or greater.

In certain embodiments, the composition may be stored for a desired period of time before administration or other use. In one embodiment, the composition may be stored for about one week, about two weeks, about three weeks, about four weeks, about five weeks or about six weeks or more, in each case retaining substantial activity (e.g., about 70%, about 75% about 80%, about 85%, about 90%, about 95%, about 98% or about 99% or more of its original activity),

In one embodiment, the composition may be stored for about one month, about two months, about three months, about four months, about five months or about six months or more. in each case retaining substantial activity (e.g., about 70%, about 75% about 80%, about 85%, about 90%, about 95%, about 98% or about 99% or more of its original activity)

### Kits

Also disclosed herein are kits comprising one or more containers filled with the compositions disclosed herein. The kits may include, for instance, containers filled with an appropriate amount of the composition disclosed herein, either as a powder, to be dissolved, or as a sterile solution.

The kit may further comprise instructional materials and, optionally, other information.

### EXAMPLES

### Example 1: Obtaining Aged Peripheral Blood

Peripheral blood is obtained from a human greater than about 40 years of age according to known methods. Mononuclear cells are isolated from fresh blood by density gradient centrifugation. Cells can be used immediately or frozen for long-term storage. The secretome of the mononuclear cells is obtained by any suitable method.

### Example 2: Obtaining Umbilical Cord Blood

Umbilical cord blood is obtained from by venipuncture of the severed umbilical cord, followed by gravity drainage into a standard sterile anticoagulant-filled blood bag using a closed system similar to the one used for whole blood collection.

### Example 3: Pre-Priming Umbilical Cord Blood Cells

Umbilical cord blood cells obtained in Example 2 are pre-primed by exposure to the secretome of the aged mononuclear cells obtained in Example 1. The exposure is between about 36 and 48 hours. The secretome of the pre-primed UBC cells is obtained by any suitable method.

### Example 4: Assessing Immune Response following Revitalization—Mitogen & Antigen Stimulation of Peripheral Blood MNC Protocol

The immune response following revitalization methods as described herein is assed using the following protocol.
Culture Medium: RPMI 1640, FBS (10%), Penicillin/Streptomycin (1%)
Controls: Numerous healthy controls are utilized, matched by age and sex.
Cell Preparation & Procedure:
1. Mononuclear cells (MNC) are isolated from heparinized peripheral blood by Ficoll Hypaque density gradient.
2. MNC are resuspended @ 106 cells/mL.
3. The following guidelines are used to label to appropriate number of sterile 17×75 cm2 tubes (1 tube/mitogen).
a. 1 tube for unstimulated or background proliferation.
4. 1 mL cell suspension/tube.
5. The appropriate volume of mitogen is added. The working concentration of each mitogen is listed below and also written on a paper placed inside the box of mitogens.
6. A 96-well plate diagram is used to map the assay. Triplicate wells are labeled for each tube.
7. 0.2 mL of cell suspension is added to 96-well flat-bottomed plates.
8. Incubation is permitted for 48 h.
9. Each well is pulsed with luCi (1 drop @ 1/50 dil) of 3HTdR.
10. Cells are harvest cells ˜16 h later. Scintillant is added and 3HTdR incorporation is determined using a scintillation counter.
Antigens that are tested include Candida or Tetanus.
The results of the assays indicate that the immune response is revitalized and in particular, in response to a control.

### Example 5: Optimization of Microvesicle Stability & Storage

Integrity of the microvesicles during long term storage is assessed. Optimum number of microvesicles/vial is tested at temperatures varying from 080 C, −20 C and 4 C. The length of storage is assessed following freezing overnight, 1 week, 4 weeks, 3 months, 6 months, and 1 year. Efficacy of storage method is determined through hematopoietic function. The results indicate that the microvesicle composition remains stable for a desired period, and that hematopoietic function is preserved to an acceptable degree.

### Example 6: Assessing Microvesicle Stability after Shipment to Distant Site

Microvesicle stability and integrity for revitalization is assessed after shipping the product. Conditions for shipment are varied. The results indicate that the microvesicle composition remains stable and integrity is preserved during shipping.

### Example 7: Assessing Optimum Size of Microvesicles for Revitalization

The optimum size of the extracellular vesicles is assessed using a microvesicle fractionation and isolation kit (e.g., from Izon LTD.) Fractionated samples are utilized to assess differences in revitalization utilizing a method disclosed herein.

### Example 8: Assess Optimum Aged Hematopoietic Source

Aged plasma, aged peripheral cells and aged bone are utilized as a source of mononuclear cells in the methods disclosed herein. Efficacy of revitalization is assessed utilizing a method disclosed herein.

### Example 9: Injection of Pre-primed Secretome into Aged-Animal Models

Aged-animal models are used to assess the degree of revitalization following injection of pre-primed EVCs (including microvesicles, exosomes or a combination thereof. Revitalization is assed short-term and long-term. Revitalization is evident both short-term and long-term, as assessed by a method disclosed herein.

### Example 10: Model for Revitalization in Human Aged Bone Marrow

A dose-response and time-course optimization for the injection of pre-primed secretome in aged human bone marrow samples is carried out. The degree of revitalization is assessed by function and phenotype. The result is validated in humanized mice.

Assessment of revitalization is performed through LT-HSC/Cobblestone Assay. Additionally, degree of revitalization is assessed through flow cytometry to identify markers associated with aging and lymphoid/myeloid bias to determine changes in hematopoietic proliferative potential. Examples of aged-associated markers include: CD41 (integrin associated with aging), Selectin P (SELP), & Clusterin (Clu).

Assessment of revitalization is performed by assessing degree of inflammation by measuring the expression of inflammatory cytokines.

Assessment of revitalization is assessed through ChiP single-cell sequencing to ensure that aged clones are no longer proliferating.

### Example 11: Batch Standardization Through Omics Studies

Proteomic and metabolomics studies are performed to assess changes in the aged hematopoietic system following revitalization.

### Example 12: In Vivo Studies of Exemplary Compositions

Aged mice were used to test the exemplary compositions comprising exosomes and to assess the ability the compositions to restore the hematopoietic system, as well as T-stem cells. Intravenous injections of the exemplary exosomes to middle—(10 month) and aged—(20 months) mice (C57bl/6) on days 0, 3 and 7. Changes were assessed as described below.

### Materials and Methods

### I. Colony Forming Units—Recombinant Human Granulocyte/Macrophage Colony Stimulating Factor (CFU rhGM-CSF)/Erythropoietin (CFU-E) using Aged-Mobilized Peripheral Blood Cells (Aged-MPBs)

### A. Materials:

a. Methylcellulose (2.7%), previously prepared and stored at −20° C. (4000 centipose—Fisher Scientific, Catalog #M-352)
b. Aged MPB, acquired from HemaCare. Total nucleated cells (TNC) previously cryopreserved in liquid nitrogen at 50E6 cells/ml
c. Umbilical cord blood (UCB), TNC previously cryopreserved in liquid nitrogen
d. Fetal Bovine Serum (FBS), batch tested to minimize variability among experiments (Atlanta biologicals, Catalog #S11150)
e. Deionized Bovine Serum Albumin (BSA), Fraction V, lyophilized, Batch tested. Prepare ˜200 mL and filter sterilize prior to storing at 4° C. (United States Biochemical Corp, Catalog #10857)
f. Beta Mercaptoethanol (14.2M). Sock will be diluted to a final concentration to 2E-4 M in methylcellulose
g. Iscoves Media (Millipore Sigma, Catalogue #13390)
h. L-glutamine, 200 mM (Millipore Sigma, Catalogue #G2510)
i. Penicilin/stroptomicin solution (P-S), 10,000 units/10 mg (Millipore Sigma, Catalogue #0781)
j. For CFU-GM Culture Media:
   i. Recombinant (r) human (h) GM-CSF (R&D systems, Catalog #215-GM)
k. For CFU-E Culture Media:
   i. hIL-3, 1.4×10³ U/ml. batch tested (Genetics Institute)
      1. Dilute stock with PBS+2% BSA. Do not freeze-thaw aliquots more than 3 times.
   ii. EPO, 500 U/ml-Please reference cytokine book for product sheet.
l. 100 mm non-adherent tissue culture plates
m. Cell equilibration media: RPMI containing 40% human serum albumin (HAS), 1% P-S, 1% L-Glutamine; add 300 Units/mL of DNase I immediately prior to use
n. 35×10 mm tissue culture plates
o. >12 ml round bottom tube
p. 3 ml syringe
   q. 16¾ gauge needle

### B. Preparation of Culture Medium:

a. CFU-GM Culture Media:

| | Final | | |
|---|---|---|---|
| Components: | Concentration: | Volumes: | |
| FBS | 30% | 36 | mL |
| BSA | 2% | 12 | mL |
| Beta Mercaptoethanol | 2E−4M | 1.2 | mL |
| rhGM-CSF | 4 U/1.65 mL | 10 | uL |
| Iscove's Media | | 18.5 | mL |
| P—S | | 0.7 | mL |
| L-Glutamine | | 0.7 | mL |
| Total Volume | | 67.2 | mL |

b. CFU-E Culture Media:

| | Final | | |
|---|---|---|---|
| Components: | Concentration: | Volumes: | |
| FBS | 30% | 36 | mL |
| BSA | 2% | 12 | mL |
| Beta Mercaptoethanol | 2 × 10⁻⁴M | 1.2 | mL |
| hIL-3 | 5 U/1.65 mL | 10 | uL |
| Epo | 6 U/1.65 mL | 500 | uL |
| Iscove's Media | | 18.0 | mL |
| P—S | | 0.7 | mL |
| L-Glutamine | | 0.7 | mL |
| Total Volume | | 67.2 | mL |

### C. Preparation of Aged-MPB Restoration:

a. Day 1: Aged-MPBs are removed from liquid nitrogen and placed into 100 mm non-adherent tissue culture plates with 10 ml equilibration media.
   i. Each cryopreserved vial is split into two non-adherent tissue culture plates with ˜25E6 cells/plate, and allowed to equilibrate for 24 hours at 37° C./5% CO₂
b. Day 2: 2E6 UCB cells are removed from liquid nitrogen and placed into non-adherent tissue culture plates with 10 ml equilibration media, and allowed to equilibrate for ˜12 hours in conditioned media at 37° C./5% CO₂
c. Upon completion of the UCB equilibration process, each the Aged-MPB and UCB are centrifuged at 300×g for 10 minutes.
   i. The Aged MPB secretome is collected, having removed the cellular components.
   ii. UCB cells are resuspended in the Aged MPB's acellular secretome.
   iii. Resuspended UCB are returned to their non-adherent tissue culture plates and incubated for 48 hours at 37° C./5% CO₂.
d. Day 3: Thaw and equilibrate a fresh tube of Aged-MPBs into non-adherent tissue culture plates as per Day 1
e. Day 4: Secretome from pre-primed UCBs is now collected and subjected to centrifugation @300×g for 10 minutes.
   i. Acellular pre-primed UCB-secretome is now administered to the Aged-MPBs that were thawed on Day 3, and incubated for 24 hours.
   ii. Like on Day 2, the Aged-MPBs thawed on Day 3 should be spun down @ 300×g for 10 minutes so they too can be resuspended in the pre-primed UCB secretome.
f. Day 5: After 24 hours, the Aged-MPBs are collected and subjected to centrifugation @ 300×g for 10 minutes.
   i. Aged-MPB cells are resuspended in 0.3 mL of CFU-GM conditioned media at a concentration of 2E6/mL.
   ii. Add 1.4 mL of 2.7% methylcellulose to a round-bottom tube using a 3 mL syringe equipped with a 16¾ gauge needle.
   iii. Add 1.5 mL of CFU-GM conditioned media to the round-bottom tube and allow natural slow mixing to occur between media and methylcellulose.
   iv. Finally, add the 0.3 mL of Aged-MPBs resuspended in CFU-GM conditioned media into the tube for subsequent mixing/homogenization.
      1. First, gently mechanically mix the contents using the syringe and needle, being sure to the minimize formation of bubbles as they can occlude colonies during counting.
      2. After mechanically mixing the contents, mix the sample by pipetting up and down with the syringe until the mixture is homogenous.
   v. Load 3 mL of methylcellulose+cell mixture loaded into the 3 mL syringe, and transfer 1.5 mL into two 35×10 mm tissue culture plates at the center of the plate.
      1. Do not tilt plate to achieve complete plate coverage, apply a weak force in a clockwise/counterclockwise rotation to uniformly distribute the semisolid media in the plate.
      2. Place six 35×10 mm tissue culture plates into larger 150×15 mm plates with a lid. Place another 35×10 mm tissue culture plate filled with sterile ddH2O to prevent evaporation from the semisolid media in the smaller plates.
   vi. Place the 150×15 mm plates into a tissue culture incubator (37 C) for 10 days and count colonies under a microscope.
      1. Check the cells at Day 6/7 to ensure there is adequate colony growth.
      2. Colonies will be counted if they appear large, clear, and greater than 20 cells/cluster. The 35×10 mm tissue culture plates should be counted on a slightly large base plate with a clear grid system so that cells can be counted in quadrants. Colonies were counted at a magnification of 4×.
         D. Isolation & Preparation of Mouse Hematopoietic Cells from Femoral Bone Marrow for CFU-GM & CFU-E Assays and Peripheral Blood for Flow Cytometry:
a. Isolation of hematopoietic cells from femoral bone marrow & Peripheral Blood for flow cytometry:
   i. Materials:
      1. Isoflurane
         a. Utilized for anesthetization of mice before euthanizing them by cardiac puncture.
      2. 35×10 mm tissue culture plates filled with PBS/RPMI for the collection and storage of femurs until researcher is ready to dissect them in a tissue culture hood.
      3. 27½ G Needles
         a. This needle will be used to draw blood via cardiac puncture and to drain hematopoietic cells from the femoral bone marrow.
      4. 1 mL Syringes
         a. This syringe will be used to draw blood via cardiac puncture and to drain hematopoietic cells from the femoral bone marrow.
      5. Sterile/Autoclaved PBS to flush femoral bone marrow.
      6. 15 mL centrifuge tubes
         a. These will be filled with 500 uL of 50 U/mL heparin to prevent the coagulation of murine peripheral blood during collection.
      7. Sterile surgical scissors
         a. These will be used to access the thoracic cavity so that murine blood can be isolated via cardiac tamponade.
      8. Sterile Forceps
         a. These will be used to hold open the thoracic cavity or to shift the heart for improved visibility of the murine heart.
   ii. Euthanasia of mice through cardiac puncture, Collection of Murine Peripheral Blood, & Collection of hematopoietic cells from Murine Femurs:
      1. Anesthetize mice by transferring them from cages to bin filled with paper towels covered in isoflurane.
         a. Transfer mice by the tail using large forceps.
      2. Transfer mouse to surgical bench with large forceps.
      3. Using surgical scissors, access the thoracic cavity of the mouse to visualize the heart.
      4. Using a 1 mL syringe equipped with a 27½ G needle, insert the needle into the left ventricle. Draw as much blood as possible and transfer into the appropriate heparin-filled 15 mL centrifgure tube.
      5. Begin the femur collection process by removing the skin and muscle from the area surrounding the femur with a sterile scalpel and surgical scissors.
      6. Carefully dislocate the femur bone from the hip and subsequently from the knee joint.
         a. Handle carefully as you do not want the bone to break longitudinally. This will make it difficult to flush the entire marrow.
      7. Transfer the femur bones into 35×10 mm tissue culture plates filled with sterile PBS and transfer plates to a tissue culture hood.
         a. Before transferring dishes to the tissue culture hood, properly package and murine carcass.
      8. Using a sterile scalpel, cut along the epiphysis at each end of the bone so that a 27½ G needle can be inserted into the body of the femur.
      9. Using a 1 mL syringe filled with sterile PBS, flush the femoral marrow into a 15 mL centrifuge tube filled with 500 uL of Dulbecco's Modified Eagles Medium (DMEM)-High Glucose.
      10. Subject the microcentrifuge tubes to centrifugation @ 300×g for 7 minutes.
   iii. Preparation of Murine hematopoietic cells for CFU-GM & CFU-E Assays:
      1. Resuspend the murine hematopoietic cells in 300 uL CFU-GM or CFU-E assay media.
      2. Using a hemocytometer, calculate the concentration of cells in each sample. Transfer 300,000 cells into the methylcellulose+CFU-GM/CFU-E assay media bacterial centrifuge tubes.
      3. Homogenize cells within tubes and mixture using a 3 cc syringe loaded with a 16¾ G needles.
      4. Using the 3 cc syringe, transfer 1.5 mL of the homogenized mixture into two 35×10 mm tissue culture plates.
         a. Place another 35×10 mm tissue culture plate filled with ddH20 in the center of a 150×15 mm plate with a lid.
      5. Colonies should be counted on:
         a. CFU-GM-Day 10, large, clear colonies containing >20 cells.
         b. CFU-E-Day 8, yellow, light pink to red colonies >20 cells.
   iv. Flow Cytometry of murine blood:
      1. Flow cytometric acquisition and analysis was carried out.

### II. Differential Ultracentrifugation & Nanosight Tracking Analysis of WR Proprietary Exosomes

A. Preparation of Sample before Differential-Ultracentrifugation (D-UC):
1. Collect culture media in a sterile 50 mL centrifuge tube.
2. Subject sample media to centrifugation @ 300×g for 10 minutes.
   a. Transfer supernatant to a new 50 mL centrifuge tube and discard of pelleted debris.
   b. At this time, turn on the bacterial centrifuge and allow the machine to come down to 4 C.
   c. At this time also move ultracentrifuge rotors to a 4 C refrigerator to reduce production of frictional heat in the ultracentrifuge.
3. Subject transferred supernatant to centrifugation @2000×g for 20 minutes.
   a. Transfer supernatant to a new 50 mL bacterial centrifuge tube and discard of pelleted debris.
4. Subject transferred supernatant to centrifugation @ 10,000×g for 30 minutes.
   a. Note: no brake was applied to slow down the rotor. It was allowed to come to a stop on its own.
5. Transfer the supernatant to 30 mL Ultracentrifuge (UC) tubes using a cotton-filled transfer pipette and rubber bulb/pipette gun.
   a. Any unoccupied volume in the tubes can be filled with 0.2 um filtered PBS.
6. Subject the 30 mL UC tubes to ultracentrifugation @ 100,000×g for 1 hour and 20 minutes at 4C.
   a. Select rotor code S30, turn on the vacuum, and begin the first D-UC spin.
7. Upon completion of the first D-UC spin, carefully aspirate the supernatant from each 30 mL UC tube. Resuspend the exosome pellet in 5 mL of 0.2 um filtered PBS and transfer the resuspended sample to a 5 mL UC tube.
   a. Note: Be sure to avoid disruption of microvesicles in the pellet.
   b. Note: Take caution when handling the cotton-filled transfer pipette as the small neck of the tubes can cause the transfer pipette to snap and contaminate your sample.
8. Now that the sample is resuspended in 5 mL UC tubes, place the tubes in the appropriate S50 rotor and change the rotor code on the machine accordingly.
   a. Subject the 5 mL UC tube to ultracentrifugation @ 120,000×g for 1 hour and 20 minutes at 4 C.
9. Upon completion of the second D-UC spin, carefully aspirate the supernatant from each 5 mL UC tube. Resuspend the exosome pellet in 0.2-1.0 mL of 0.2 um filtered PBS and transfer the resuspended sample to a sterile 2 mL centrifuge tube.
   a. The final resuspension volume will be dependent on the specific downstream cellular assay. It is always better to dilute less initially and adjust volumes later.

### B. Nanosight Tracking Analysis (NTA) of Microvesicles/Exosomes Using NS300:

1. Dilute your exosome sample in a 1:10E4 dilution.
   b. Note: The NS300 machine is optimized to record nanoparticle counts in the 10E6-10E10 range. Thus, you can adjust your dilution depending on how concentrated your sample is on the machine.
   c. Very concentrated samples will be reported as “High Noise” on the machine and a inadequately concentrated sample will be reported as “Low Tracks <200” or “Low Tracks <100”.
2. Select the NS300 program on the computer and turn on the NS300 device.
   a. Note: The power button should be turned on before opening the program. If opened incorrectly, the program will sometimes report errors with detecting the camera/temperature control. These errors can be fixed by restarting the machine and software, in that respective order.
3. Once the program is properly running, clean the NS300 with ddH2O using a 1 ml syringe.
   a. Note: Clean the machine with 4 mL of water or until there is minimal debris detected by the 488 nm camera.
   b. Note: You should also set the “User-Lines” to 30 and 150 nm as exclusion bars so that anything above and below those respective sizes will be excluded.
4. After cleaning the NS300, an SOP can be opened on the program. Set the SOP to 5 captures with 60 second capture durations. Set the syringe pump flow to continuous at 100. Finally, report the correct dilution as this value will be used to adjust your final particle concentration on the final NTA report.
   a. After reading the first sample, wash the NS300 with ddH2O 4 times to ensure that debris does not get recorded in the following sample.
5. Open NTA reports to view nanoparticle concentration and purity of sample.

### III. Mixed Lymphocyte Reaction

Principle: This reaction will measure the response of both the MHC and non-MHC restricted response to non-cycling stimulator cells.

### A. Materials:

1. Assay Medium: RPMI (89%), FBS (10%), Beta-mercaoptoethanol 3E-3M (1%)
2. Wash Medium: RPMI (98%), FBS (2%)
3. Plates: 96-well flat bottomed plates.

### B. Mononuclear Cell (MNC) Preparation:

1. Separate MNC from peripheral blood of responders and stimulators by Ficoll Hypaque density gradient.
2. Adjust responders to 2E6 cells/mL in assay medium.
3. Adjust stimulator cells to ˜10E6 cells/mL in wash medium.
   a. Note: The stimulator cells should be brought to a concentration greater than 2E6/mL (ideally @ 10E6/mL) for subsequent irradiation. However, if the total volume must remain below 5 mL, because the 10E6 concentration is not attainable due to low MNC yield, you can adjust the concentration to any value greater than 2E6 cells/ml because you will adjust the stimulator set to 2E6 cells/mL before seeding cells on the 96-well plate.

### C. Control:

1. If possible, more than one control responder should be included with each assay. This is necessary with humans to avoid the possibility of MHC match between the responder and stimulator. If this occurs, the entire assay may be misinterpreted as a technical error. A combination of control responders will indicate a MHC match.

### D. Stimulator:

Note: Because the MHC type of human is not defined, more than one stimulator should be used.

Note: Stimulators may be mixed or used individually. If mixing, add together immediately before subjecting cells to cycle inhibition.
1. Treatment: Stimulators should be placed in a non-cycling state. This is accomplished by either irradiation or mitomycin treatment.
   a. Note: WR Biotech II's proprietary technology only evaluated MNC stimulation using the irradiation method.
2. Irradiation:
   a. Using a cesium (¹³⁷ Cs) source to irradiate washed stimulator. Cells should be resuspended at ˜10E6/mL in wash medium and then irradiated with 5000 rads.
      i. Note: Check the rate of exposure in the room that contained the unit and adjust the timing of exposure depending on the reported radioactive intensity.
   b. Centrifuge cells and resuspend at 2E6 cells/mL in assay medium.
3. Cell Stimulation Controls:
   a. Unstimulated responder+Medium
   b. Non-Cycling Stimulator+Medium

### E. Cell Seeding & Setup Using a 96-Well Plate:

1. Use a flat-bottomed 96-well template and make a plate diagram. Label the plate with necessary conditions in sets of 5-7 technical replicates per set.

Note: If the results are expected to be highly variable, use more replicates to account for these differences.
2. Following the plate diagram, add 0.1 mL of both responders and stimulator cells to wells.

Note: Both responder and stimulator cells are resuspended at 2E6/mL, which translates to 2E5 cells/well. Knowing this amount, one can adjust the initial volume of blood drawn to yield the appropriate number of cells.

Note: To approximate how much blood is needed, use the following approximation to determine blood volume. 1 mL of peripheral blood yields ˜1E6 MNCs total.

Note: Before incubating the cells for 72 h, one should add any third party inflammatory mediators to the wells if pertinent to research question. WR Biotech II added their proprietary exosomes at increasing concentrations from 2E2 exosomes/mL-2E9 exosomes/mL to determine if there is any MHC-induced proliferation.
3. Incubate the cells for 72 h.

Note: Cells are left to incubate for 72 h because this is close to the peak proliferation event where 3HTdR will be used to measure proliferation depending of 3HTdR incorporation.
4. On the afternoon of day 3, pulse each well with 1 uCi (1 drop @ 1/50 dilution) of 3HTdR.
5. Harvest cells ˜16 h later. Using cell harvester.

Note: Allow the hole punched membranes to dry over night before adding scintillation fluid.
6. Add 5 mL of scintillation fluid per scintillation vial.
7. Determine the amount of 3HTdR incorporation in a scintillation counter.

### Results

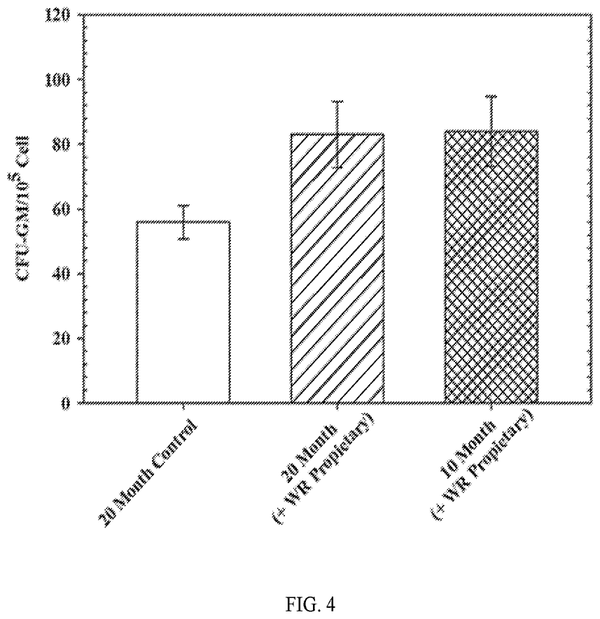

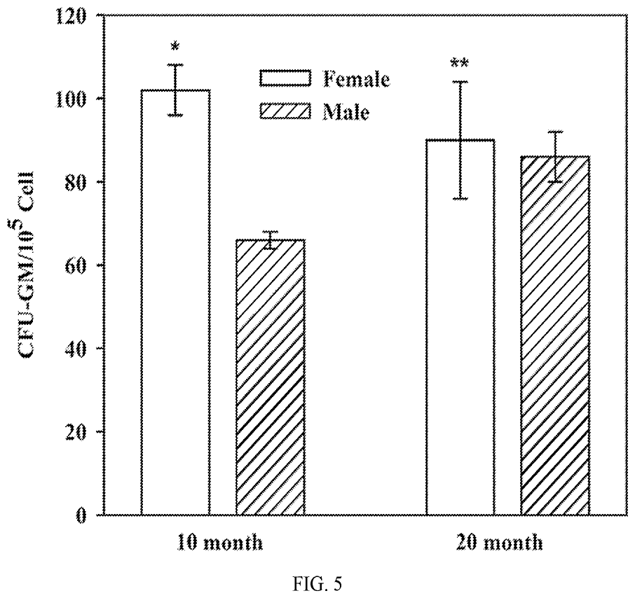

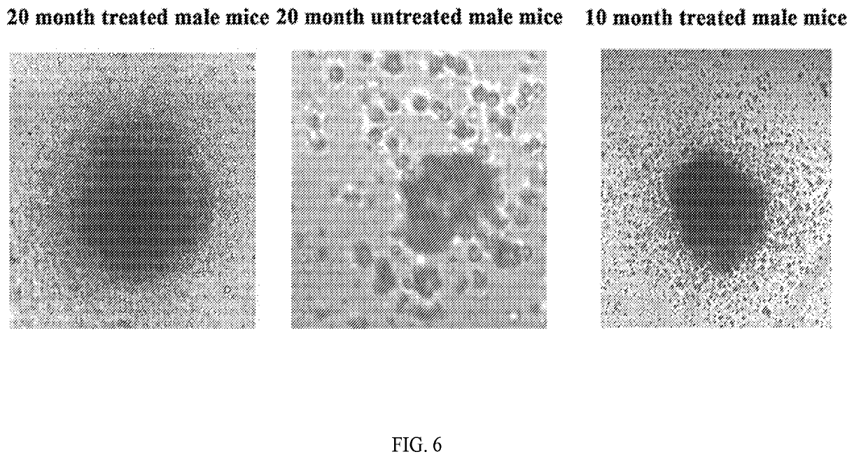

Mice (n=2), 10 and 20 months, were treated with 10¹⁰ particles of exemplary exosomes. Mice were injected intraperitoneally at Day 0 and Day 3. Mice were euthanized at Day 10. The cells in mice femurs were collected and assayed for colony forming unit (CFU-GM). The results are shown in FIGS. 4 and 5. Images of relative clonogenic size from bone marrow cells are shown in FIG. 6.

It was observed that the mice responded rapidly to the exemplary exosomes by increased hematopoietic colonies. The colonies were larger than those from untreated mice. There was a delay in 10-month old male mice with respect to hematopoietic restoration. Behaviorally, the treated mice were active and appeared responsive as young healthy mice after 3 days of the first injection.

### Example 13: Exemplary Exosomes from Aged Plasma

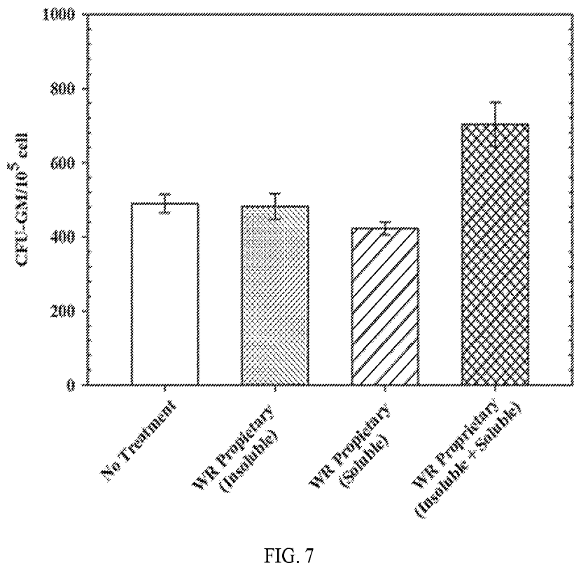

Aged plasma was used to isolate exosomes to assess whether other aged tissues can cause the umbilical cord blood cells to release restorative exosomes. The exosomes from aged plasma were added to aged mobilized peripheral blood (MPB). Clonogenic assays for granulocyte-macrophage progenitors (CFU-GM) were subsequently performed, as described above. The results, shown in FIG. 7, were measured for insoluble and soluble fractions of the samples.

It was determined that exosomes from the aged plasma were unable to enhance hematopoietic restoration of the aged cells (Aged-Plasma Exosomes). In contrast, the exemplary exosomes enhanced the restoration endpoint.

### Example 14: Stability Assessment of Exemplary Compositions

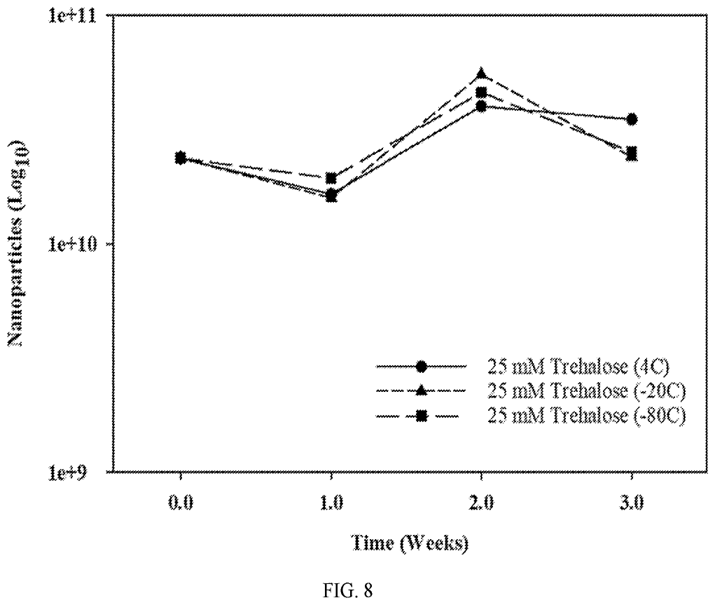

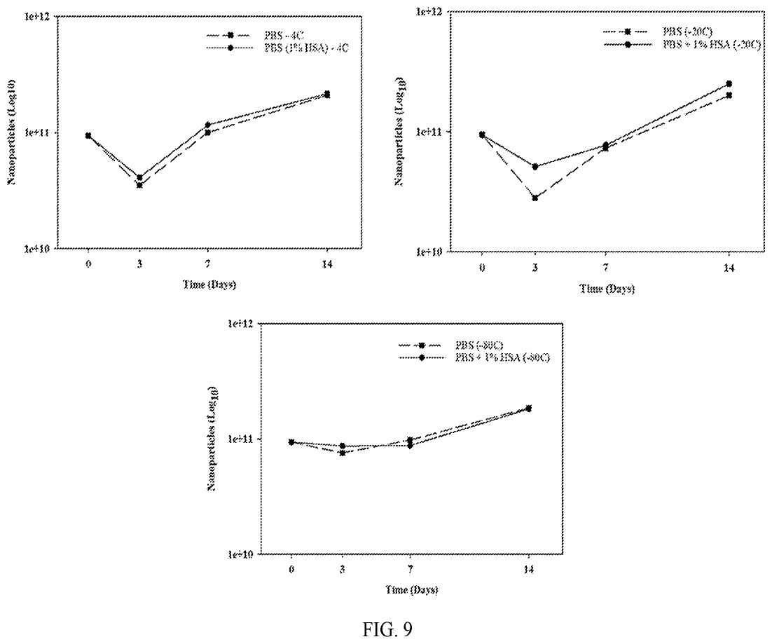

Various cryopreservation techniques were used to assess short-and long-term stability of the exosomes. Exosomes were stored at various temperatures (4° C., −20° C., and −80° C.) with different freezing components (phosphate buffered saline (PBS) +/−trehalose (a sugar) or human serum albumin (HSA; a protein)). The results are shown in FIGS. 8-11.

Particles were enumerated by Nanosight Tracking Analysis (NTA). The nanosight's optimal range to quantitiate exosomes ranged between 10⁶ and 10^{9.} Thus, the small variability noted in the graphs are negligible.

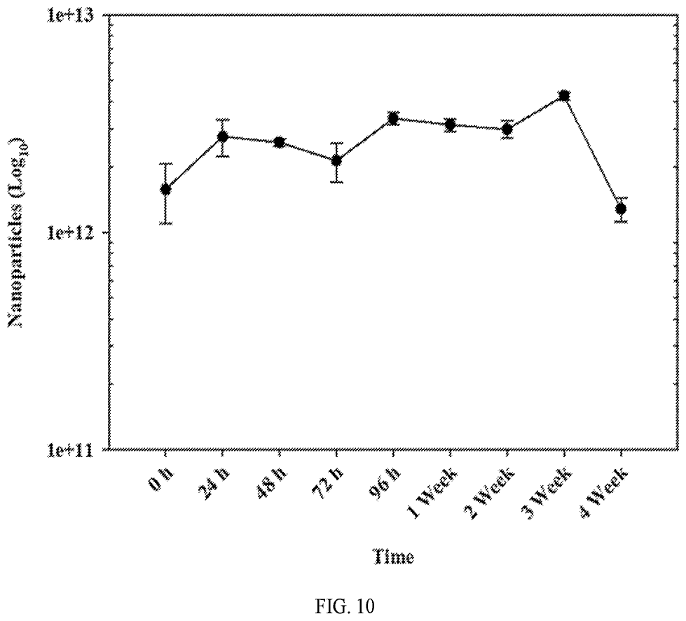

The optimized conditions of PBS at 4° C. were used to assess the stability of WRB's proprietary exosome preparation at higher concentrations to ensure maintenance of stability (FIG. 10).

It was observed that the stability of the exosomes was relatively constant at all temperatures. Generally, cryopreserved exosomes were found to be highly stable and consistent across the tested temperature conditions. Addition of HSA did not increase cryopreservation stability as compared to PBS alone in short-term storage. Although, a drop in nanoparticle count was observed at Week 4, this particle count is relatively consistent when compared to baseline. Overall, the stability of the exemplary exosomes were consistent across both short-and long-term time courses.

What is claimed is:

1. An allogeneic, acellular composition comprising extracellular vesicles prepared by a process comprising:
(a) incubating mononuclear cells in a cell culture media for a period of 12-24 hours, said mononuclear cells producing a secretome,
(b) processing the cell culture media from step (a) to remove the mononuclear cells to obtain a purified cell culture media containing the secretome of the mononuclear cells,
(c) contacting a biological sample comprising stem cells with at least a portion of the purified cell culture media containing the secretome of the mononuclear cells from step (b) for a period of 24-96 hours to produce a cell culture media comprising extracellular vesicles produced by the biological sample, and
(d) processing the cell culture media comprising extracellular vesicles produced by the biological sample comprising stem cells obtained in step (c) to remove cells to obtain the allogeneic, acellular composition comprising extracellular vesicles,
wherein the mononuclear cells of step (a) are obtained from a subject that is older than the subject that is the source of the biological sample comprising stem cells of step (c).

2. The allogeneic, acellular composition comprising extracellular vesicles of claim 1, wherein the mononuclear cells are human mononuclear cells.

3. The allogeneic, acellular composition comprising extracellular vesicles of claim 1, wherein the biological sample comprising stem cells comprises human stem cells.

4. The allogeneic, acellular composition comprising extracellular vesicles of claim 1, wherein processing the cell culture media to remove the mononuclear cells in step (b) comprises centrifugation.

5. The allogeneic, acellular composition comprising extracellular vesicles of claim 1, wherein processing the cell culture media comprising extracellular vesicles produced by the biological sample comprising stem cells obtained in step (c) comprises centrifugation.

6. The allogenic, acellular composition comprising extracellular vesicles of claim 5, wherein the extracellular vesicles are further purified using differential centrifugation, density-gradient centrifugation, gel-permeation chromatography, affinity capture, microfluidic devices, synthetic polymer-based precipitation, or membrane filtration.

7. The allogenic, acellular composition comprising extracellular vesicles of claim 6, wherein the extracellular vesicles are further purified using differential centrifugation at centrifugal accelerations of between about 19,000×g and about 100,000×g.

8. The allogeneic, acellular composition comprising extracellular vesicles of claim 1, wherein the mononuclear cells comprise peripheral blood cells, plasma cells, mobilized peripheral blood cells, or bone marrow cells.

9. The allogeneic, acellular composition comprising extracellular vesicles of claim 8, wherein the mobilized peripheral blood cells are obtained through the use of a mobilizing agent that recruits cells from the bone marrow into the blood stream.

10. The allogeneic, acellular composition comprising extracellular vesicles of claim 9, wherein the mobilizing agent is granulocyte colony stimulating factor, plerixafor, or a combination of both.

11. The allogeneic, acellular composition comprising extracellular vesicles of claim 1, wherein the biological sample comprising stem cells comprises hematopoietic stem cells.

12. The allogeneic, acellular composition comprising extracellular vesicles of claim 1, wherein the biological sample comprising stem cells comprises umbilical cord blood cells.

13. The allogeneic, acellular composition comprising extracellular vesicles of claim 1, wherein the biological sample comprising stem cells comprises mammary stem cells, intestinal stem cells, mesenchymal stem cells, endothelial stem cells, neural stem cells, olfactory stem cells, neural crest stem cells, placental cells, or testicular cells.

14. The allogeneic, acellular composition comprising extracellular vesicles of claim 1, wherein the biological sample comprising stem cells comprises cells obtained from bone marrow, adipose tissue, peripheral blood, placental blood, extraembryonic membranes, or placenta.

15. The allogeneic, acellular composition comprising extracellular vesicles of claim 1, wherein the extracellular vesicles include microvesicles, exosomes or a combination thereof.

16. A pharmaceutical composition comprising the allogeneic, acellular composition comprising extracellular vesicles of claim 1, wherein the composition comprises a population of from about one million to about five million extracellular vesicles in a volume of about 200 microliters to about 500 microliters.

17. The allogeneic, acellular composition comprising extracellular vesicles of claim 1, wherein mononuclear cells that are contacted with the composition exhibit decreased expression of CD41, Selectin P, or Clusterin, as compared to mononuclear cells that are not contacted with the composition.

18. The allogeneic, acellular composition comprising extracellular vesicles of claim 1, wherein mononuclear cells that are contacted with the composition exhibit decreased expression of interleukin-1, interleukin-6, interleukin-18, tumor necrosis factor alpha, or interferon gamma, as compared to mononuclear cells that are not contacted with the composition.

19. The allogeneic, acellular composition comprising extracellular vesicles of claim 1, wherein plasma from a subject that is treated with the composition contains reduced levels of interleukin-1, interleukin-6, interleukin-18, tumor necrosis factor alpha, or interferon gamma, as compared to plasma from a subject that is not treated with the composition.

20. The allogeneic, acellular composition comprising extracellular vesicles of claim 1, wherein cells that are contacted with the composition exhibit decreased expression of FGFR1, MET, or NTRK1, or increased expression of PTEN, as compared to cells that are not contacted with the composition.

21. The allogeneic, acellular composition comprising extracellular vesicles of claim 1, wherein cells that are contacted with the composition exhibit decreased expression of C-reactive protein or troponin, as compared to cells that are not contacted with the composition.

22. The allogeneic, acellular composition comprising extracellular vesicles of claim 1, wherein cells that are contacted with the composition exhibit decreased expression of isoprostanes, tau, AB, sulphatides, homocysteine, or combinations thereof, as compared to cells that are not contacted with the composition.

23. The allogeneic, acellular composition comprising extracellular vesicles of claim 1, wherein hematopoietic cells that are contacted with the composition exhibit increased proliferation or differentiation as assayed by a colony-forming unit (CFU) assay, as compared to hematopoietic cells that are not contacted with the composition.

24. The allogeneic, acellular composition comprising extracellular vesicles of claim 1, wherein hematopoietic cells that are contacted with the composition exhibit increased proliferation or differentiation as assayed by a LT-HSC/Cobblestone Assay, as compared to hematopoietic cells that are not contacted with the composition.

25. A method of treating a cardiovascular disorder, a neurological disorder, or an immunologic disorder that is amenable to treatment by the allogeneic, acellular composition comprising extracellular vesicles of claim 1 in a subject in need thereof, comprising administering a therapeutically effective amount of the allogeneic, acellular composition comprising extracellular vesicles prepared by the process of claim 1 to said subject.

26. The method of claim 24, wherein the disease or disorder is a disease or disorder associated with aging.

27. A method of producing an allogeneic, acellular composition comprising extracellular vesicles, said method comprising:
(c) incubating mononuclear cells in a cell culture media for a period of 12-24 hours, said mononuclear cells producing a secretome,
(d) processing the cell culture media from step (a) to remove the mononuclear cells to obtain a purified cell culture media containing the secretome of the mononuclear cells,
(c) contacting a biological sample comprising stem cells with at least a portion of the purified cell culture media containing the secretome of the mononuclear cells from step (b) for a period of 24-96 hours to produce a cell culture media comprising extracellular vesicles produced by the biological sample, and
(d) processing the cell culture media comprising extracellular vesicles produced by the biological sample comprising stem cells obtained in step (c) to remove cells to obtain the allogeneic, acellular composition comprising extracellular vesicles,
wherein the mononuclear cells of step (a) are obtained from a subject that is older than the subject that is the source of the biological sample comprising stem cells of step (c).

28. The method of claim 27, wherein the mononuclear cells comprise human mononuclear cells.

29. The method of claim 27, wherein the biological sample comprising stem cells comprises human stem cells.

30. The method of claim 27, wherein processing the cell culture media to remove the mononuclear cells in step (b) comprises centrifugation.

31. The method of claim 27, wherein processing the cell culture media comprising extracellular vesicles produced by the biological sample comprising stem cells obtained in step (c) comprises centrifugation.

32. The method of claim 31, wherein the extracellular vesicles are further purified using differential centrifugation, density-gradient centrifugation, gel-permeation chromatography, affinity capture, microfluidic devices, synthetic polymer-based precipitation, or membrane filtration.

33. The method of claim 32, wherein the extracellular vesicles are further purified using differential centrifugation at centrifugal accelerations of between about 19,000×g and about 100,000×g.

34. The method of claim 27, wherein the mononuclear cells comprise peripheral blood cells, plasma cells, mobilized peripheral blood cells, or bone marrow cells.

35. The method of claim 34, wherein the mobilized peripheral blood cells are obtained through the use of a mobilizing agent that recruits cells from the bone marrow into the blood stream.

36. The method of claim 35, wherein the mobilizing agent is granulocyte colony stimulating factor, plerixafor, or a combination of both.

37. The method of claim 27, wherein the biological sample comprising stem cells comprises hematopoietic stem cells.

38. The method of claim 27, wherein the biological sample comprising stem cells comprises umbilical cord blood cells.

39. The method of claim 27, wherein the biological sample comprising stem cells comprises mammary stem cells, intestinal stem cells, mesenchymal stem cells, endothelial stem cells, neural stem cells, olfactory stem cells, neural crest stem cells, placental cells, or testicular cells.

40. The allogeneic, acellular composition comprising extracellular vesicles prepared by the process of claim 1, wherein the biological sample comprising stem cells comprises cells obtained from bone marrow, adipose tissue, peripheral blood, placental blood, extraembryonic membranes, or placenta.

41. The method of claim 27, wherein the extracellular vesicles include microvesicles, exosomes or a combination thereof.
